(19) United States
(12) Patent Application Publication
(10) Pub. No.: US 2024/0058505 A1
(43) Pub. Date: Feb. 22, 2024

(54) CONDUCTIVE SCAFFOLDS FORMED BY ABSORBABLE COMPOSITE BIOMATERIALS AND USE THEREOF

(71) Applicant: CONDUCTIVEBIO INCORPORATED, Santa Clara, CA (US)

(72) Inventors: Michael SPAID, Mountain View, CA (US); Jeffrey WOLK, Half Moon Bay, CA (US); Haixia DAI, Pleasanton, CA (US); Pierre-Marc ALLEMAND, Santa Clara, CA (US); Pin Chu CHEN, San Jose, CA (US); Ian MOODY, Soquel, CA (US); Michael V. PAUKSHTO, Foster City, CA (US)

(21) Appl. No.: 18/259,252

(22) PCT Filed: Dec. 22, 2021
(86) PCT No.: PCT/US2021/064915

§ 371 Date: Jun. 23, 2023

Related U.S. Application Data
(60) Provisional application No. 63/130,570, filed on Dec. 24, 2020.

(51) Int. Cl. A61L 27/24; A61L 27/04; A61N 1/05; A61L 31/04; A61L 31/02; A61L 27/50; A61L 31/14

(52) U.S. Cl. CPC A61L 27/24; A61L 27/047; A61N 1/0551; A61L 31/044; A61L 31/022; A61L 27/50; A61L 31/14; A61L 2400/12; A61L 2430/32

(57) ABSTRACT

Provided herein are electrically conductive scaffolds of various shapes suitable for promoting and stimulating tissue regeneration, particularly in nerve repair.

### BACKGROUND

### Technical Field

The present disclosure relates to biocompatible and bio-absorbable scaffolds suitable for facilitating or stimulating tissue repair or regeneration, or modulating tissue response to an injury or disease.

### Description of the Related Art

Implantable scaffolds are capable of supporting and guiding tissue repair or regeneration at sites of tissue damage caused by disease, injury or congenital abnormalities. Certain structural features of the scaffolds can play an important role in facilitating cellular adhesion, migration, and organization, which are necessary cellular responses for tissue regeneration to replace those lost due to the damage. In particular, for oriented tissues such as nerves, anisotropic scaffolds can be advantageous in guiding the alignment of the regenerated tissue.

Electrically conductive scaffolds have been explored as a means to stimulate tissue response which may further promote tissue regeneration or reduce pain and other chronic conditions. Electrospun conductive polymers as well as silk coated with graphene oxide are known materials for fabricating electrically conductive scaffolds.

### BRIEF SUMMARY

Provided herein are electrically conductive scaffolds made of composite biomaterials of one or more biocompatible polymers incorporated with a mesh of nanostructures of conductive or semiconductive materials.

The implantable scaffolds are electrically conductive owing to the conductive mesh. The conductive nanostructures are typically anisotropically shaped structures (e.g., nanowires, nanotubes or nanoribbons) of metals or semiconductors. The nanostructures have high aspect ratios and readily reach the percolation threshold (i.e., long range connectivity) needed to form a conductive network or mesh. The conductivity is adjustable based on the density of the interconnected conductive network of nanostructures.

Biocompatible polymers, preferably fibrillar biopolymers, combine the mechanical strength and orientational anisotropy needed for the scaffolds to act as the structural support for cell attachment, cell alignment and subsequent tissue development. In various embodiments, the fibrillar biopolymers are derived from polymeric materials naturally rich with long and organized nanofibrils, such as collagen and chitin.

The fibrillar biopolymers, with optional physical or chemical modifications, are highly processable, allowing the electrically conductive scaffolds to take any dimensions and shapes, depending on the end applications.

The electrically conductive scaffolds have versatile applications, particularly in tissue engineering and regenerative medicine. Combining the scaffolding and electrical stimulation that regulates or manipulates cell behaviors, the electrically conductive scaffolds are useful for nerve repair or heart tissue repair following myocardial infarction. See, e.g., Langmuir 29(35) 11109-11117 (2013), Biomaterials Research 23:25, (2019). In various embodiments, they provide electrical stimulation to facilitate rehabilitation of patients with stroke, Alzheimer's, glioblastoma, etc. In various embodiments, the electrically conductive scaffolds may also be implanted as neural tissue interface, drug release depot, or as image contrast agents due to their radio opacity or conductivity (e.g., CT tomography or MM). In further embodiments, they may also be used as cell transfer scaffolds with in-vitro preconditioning for wound healing, myocardial infarction, etc.

### BRIEF DESCRIPTION OF THE SEVERAL VIEWS OF THE DRAWINGS

FIGS. 1A-1C show schematically configurations of conductive membranes according to different embodiments.

FIGS. 2A-2C are specific examples of the conductive membranes of the embodiments shown in FIG. 1A-1C, respectively.

FIG. 2D shows the SEM images of the conductive membrane of FIG. 2B (at different magnifications).

FIGS. 3A-3C show the effect of a collagen binder for coating silver nanowire (AgNW) layer on a substrate.

FIGS. 4A-4C are respective dark field images of conductive coatings having different relative amounts of silver nanowires and collagen binder.

FIG. 5A shows the continuous and uniform appearance of a conductive coating formed from silver nanowires and hyaluronic acid (HA) binder.

FIG. 5B shows dark field images of conductive coatings with various binders.

FIG. 6A shows a single-layer conductive tube formed by rolling the conductive membrane of FIG. 1C.

FIG. 6B shows a multi-layer conductive tube formed by rolling the conductive membrane of FIG. 1C.

FIG. 6C shows a conductive tube having in its interior space one or more threads of a fibrillar biopolymer or threads of a conductive composite fibrillar biopolymer with metal nanostructures.

FIG. 7A shows a conductive tube having a uniform conductive layer on the interior surface of a biopolymer tube.

FIG. 7B shows taking an end-to-end measurement of the resistance of the conductive tube.

FIGS. 7C and 7D show the SEM images of the interior of the conductive tube in different degrees of magnification.

FIG. 8A shows a porous collagen tubes coated with a conductive membrane (collagen/silver nanowire mesh) according to one embodiment.

FIG. 8B shows a porous collagen tubes coated with a conductive membrane (collagen/silver nanowire mesh/collagen) according to another embodiment.

FIG. 8C shows a conductive collagen cylinder prepared by rolling a conductive membrane on a rod according to one embodiment.

FIG. 9A shows a process of forming a conductive thread from a conductive membrane (as a dried film).

FIG. 9B shows taking an end-to-end measurement of a segment of the conductive thread formed according to the process shown in FIG. 9A.

FIG. 10 shows a picture of three conductive threads prepared by a method illustrated in FIG. 9A having resistance of 10 ohm/cm (right side), 54 ohm/cm (left side), and 333 ohm/cm (middle).

FIGS. 11A-11C show a thread's cross section having the nanowire mesh interleaved with collagen in a pleated format.

FIG. 11D shows silver nanowire network of the thread.

FIG. 12A shows a conductive thread formed by coating a biopolymer thread (e.g., a suture) with a layer of conductive nanostructures.

FIG. 12B shows taking an end-to-end measurement of the conductive thread of FIG. 12A.

FIGS. 13A-13C show SEM images of AgNW coated catgut sutures in increasing magnifications.

FIGS. 14A-14B show conductive scaffolds with electrical contacts.

FIG. 14C shows schematically a culture system for testing electrical stimulation in vitro on cell behavior.

FIG. 15 shows a tubular conductive scaffold as a nerve repair conduit, made of a collagen tube having a conductive insert including a conductive strip that includes a flap that makes direct contact with the proximal and distal ends of the nerve.

FIG. 16A shows a conductive dressing/bandage made of a collagen/silver nanowire mesh/collagen membrane for wound treatment.

FIG. 16B shows schematically a conductive wound dressing being applied to an open wound in skin.

FIG. 17 shows a transparent conductive dressing/bandage made of the collagen/silver nanowire mesh/collagen membrane on a face for cosmetic treatment.

FIG. 18 shows the CT images taken at 2 weeks, 5 weeks, 10 weeks, and 6 months after implanting conductive collagen threads into a rat.

FIGS. 19A-19B show the construction of a NeuraGuide™ device according to one embodiment.

FIG. 20 shows the implantation of a wireless nerve stimulator in an animal model.

FIG. 21 shows the compound nerve action potential (CNAP) for the NeuraGuide™ treatment group as compared to the NeuraGen® control group.

FIG. 22 shows the electromyography (EMG) measurements between the NeuraGuide™ treatment group as compared to the NeuraGen® control group.

FIG. 23 is a histological section taken near the distal end from an explanted NeuraGuide™ device, showing numerous myelinated axons that have bridged the nerve gap.

### DETAILED DESCRIPTION

Various embodiments provide electrically conductive scaffolds for regenerating tissue, restoring function, reducing pain or providing a support for other treatment. As used herein, “scaffold” refers to a structural support or matrix that provides a physical environment for cell attachment, proliferation and extracellular matrix deposition, followed by tissue ingrowth.

The scaffolds according to the various embodiments of this disclosure are electrically conductive because they are formed from composite biomaterials combining one or more biocompatible polymers with a mesh of nanostructures of conductive or semiconductive materials. The electrically conductive scaffolds may be implanted in vivo or used in vitro (e.g., as cell scaffolding for preconditioning cell transfer). Depending on tissue-specific considerations, the scaffolds may be suitably adapted to receive, either wired or wirelessly, a power bias or electrical current, thereby delivering electrical stimulation to the tissue or cells. In other uses, an electrical current to the scaffolds produces a Joule heating effect that may facilitate healing or cause a localized ablation of a tumor.

The electrically conductive scaffolds are biocompatible and structurally stable during the period necessary for tissue regeneration; and are ultimately absorbed by the body after being degraded, dissolved or metabolized. The conductive nanostructures are either inert/non-toxic (e.g., Pt, Au) or naturally antimicrobial (e.g., Ag). In addition, because of the minuscule amounts and sizes of the conductive nanostructures needed to form a conductive network, they pose little risk in eliciting toxic or immune response. These and other aspects of the electrically conductive scaffolds are discussed in further detail below.

### Composite Biomaterial

The composite biomaterials, according to the various embodiments, comprise one or more biocompatible polymers and a conductive mesh or network of nanostructures. The composite biomaterials, owing to the presence of the conductive network, may have functional properties of electrical and semiconductive devices. Once implanted, they are capable of interacting with external or internal energy sources.

### A. Biocompatible Polymers

Biocompatible polymer is the structural component of the composite biomaterial, contributing to the mechanical strength, pliability, porosity, and optionally orientational features to the scaffolds. As used herein, “biocompatible polymer” refers to a polymer which, in the amounts employed, is non-toxic, chemically inert, and substantially non-immunogenic when used internally in a mammalian body (e.g., a human patient). Biocompatible polymers include natural polymers, synthetic polymers, or combination thereof.

The natural polymers are biopolymers originated from or produced by cells of living organisms. Suitable biopolymer may be fibrillar or non-fibrillar. Fibrillar biopolymers have linear arrays of repeating subunits or structural motifs, forming a higher-order structure by intramolecular or intermolecular hydrogen bonding. Fibrillar biopolymers can be processed (e.g., arrayed) into various forms, providing a fibrous matrix into which cell attachments or alignment can take hold. Natural fibrillar biopolymers include, for example, collagen, fibrin, fibrinogen, fibronectin, laminin, silk, as well as modified polysaccharides such as chitin, gelatin, glycosaminoglycans (GAGs), chitosan, sodium alginate, alginic acid and the like.

In preferred embodiments, the biopolymer is collagen or a collagen derivative. Collagen is naturally fibrous, flexible and biocompatible. Collagen-based biopolymers are known to provide orientational anisotropy after having been arrayed in various forms (e.g., aligned, kinked, or woven). In particular, cross-linked pseudo-fibers converted from collagen-based films have been demonstrated to provide the strength, elasticity and guidance required for cell attachment and alignment. Detailed description of preparing, purifying and fabricating oriented collagen can be found in, for example, U.S. Pat. No. 8,513,382, which is incorporated herein by reference in its entirety.

Synthetic biocompatible polymers include, for example, polyethylene glycol (PEG), polycaprolactone (PCL), polyglycolic acid (PGA), and poly(lactide-co-glycolide) (PLGA), hydroxypropyl cellulose (HPC), hydroxypropyl methylcellulose (HPMC), and the like. These synthetic biocompatible polymers may be used alone or be combined with biopolymer.

In certain embodiments, the naturally fibrillar biopolymer may be purified and used directly to form the composite material. In other embodiments, the naturally fibrillar biopolymer may be coupled to or physically blended with one or more synthetic biocompatible polymer.

### B. Nanostructures

As used herein, “nanostructures” generally refer to electrically conductive nano-sized structures, at least one dimension of which (i.e., width or diameter) is less than 500 nm, and more typically less than 100 nm or 50 nm. In various embodiments, the width or diameter of the nanostructures are in the range of 10 to 40 nm, 20 to 40 nm, 5 to 20 nm, 10 to 30 nm, 40 to 60 nm, 50 to 70 nm.

The nanostructures can be of any shape or geometry. One way for defining the geometry of a given nanostructure is by its “aspect ratio,” which refers to the ratio of the length and the width (or diameter) of the nanostructure. In certain embodiments, the nanostructures are isotropically shaped (i.e., aspect ratio=1). Typical isotropic or substantially isotropic nanostructures include nanoparticles. In preferred embodiments, the nanostructures are anisotropically shaped (i.e., aspect ratio 1). The anisotropic nanostructure typically has a longitudinal axis along its length. Exemplary anisotropic nanostructures include nanowires (solid nanostructures having aspect ratios of at least 10, and more typically at least 50), nanorod (solid nanostructures having aspect ratio of less than 10), nanoribbons (nano thin solid flakes), and nanotubes (hollow nanostructures).

Lengthwise, anisotropic nanostructures (e.g., nanowires) are more than 500 nm, or more than 1 μm, or more than 10 μm in length. In various embodiments, the lengths of the nanostructures are in the range of 5 to 30 μm, or in the range of 15 to 50 μm, 25 to 75 μm, 30 to 60 μm, 40 to 80 μm, or 50 to 100 μm.

The nanostructures can be of any conductive or semiconductive material. More typically, the nanostructures are formed of a metallic material, including elemental metal (e.g., transition metals) or a metal compound (e.g., metal oxide). The metallic material can also be a bimetallic material or a metal alloy, which comprises two or more types of metal. Suitable metals include, but are not limited to, silver (Ag), gold (Au), palladium (Pd), platinum (Pt), iridium (Ir), magnesium (Mg), zinc (Zn), silicon (Si), germanium (Ge) or alloys thereof.

Suitable nanowires typically have aspect ratios in the range of 10 to 100,000. Larger aspect ratios can be favored for obtaining a transparent conductor layer since they may enable more efficient conductive networks to be formed while permitting lower overall density of wires for a high transparency. In addition, when conductive nanowires with high aspect ratios are used, the density of the nanowires that achieves a conductive network can be low enough that the conductive network has reduced cytotoxicity and faster biodegradation. Moreover, the diameter of nanowires can be modified for controlling the biodegradation rate. Typically, thinner nanowires have a faster biodegradation rate.

Conductive nanowires include metal nanowires and other conductive particles having high aspect ratios (e.g., higher than 10). Examples of non-metallic nanowires include, but are not limited to, carbon nanotubes (CNTs), metal oxide nanowires, conductive polymer fibers and the like.

As used herein, “metal nanowire” refers to a metallic wire comprising elemental metal, metal alloys or metal compounds (including metal oxides). At least one cross-sectional dimension of the metal nanowire is less than 500 nm, and less than 200 nm, and more preferably less than 100 nm. As noted above, the metal nanowire has an aspect ratio (length:diameter) of greater than 10, preferably greater than 50, and more preferably greater than 100. Suitable metal nanowires can be based on any metal, including without limitation, silver, gold, copper, nickel, and gold-plated silver.

The metal nanowires can be prepared by known methods in the art. In particular, silver nanowires can be synthesized through solution-phase reduction of a silver salt (e.g., silver nitrate) in the presence of a polyol (e.g., ethylene glycol) and poly(vinyl pyrrolidone). Large-scale production of silver nanowires of uniform size can be prepared according to the methods described in, for example, U.S. Pat. Nos. 10,026,518 and 10,081,058, all of which are incorporated herein by reference in their entireties.

### C. Conductive Mesh

The nanostructures in the composite material form a conductive mesh (or simply “mesh”), also referred to as a conductive network or a conductive layer. A mesh is an interconnected 2D or 3D network of conductive or semiconductive nanostructures. The “middle plane of a mesh” is the plane having the least deviation from the mesh. The “thickness of the mesh” is the maximum distance from the mesh to the middle plane. The “mesh surface loading” is the weight of the mesh materials in the infinite square cuboid which has 1 cm 2 square cross section with the middle plane.

Since conductivity is achieved by electrical charge percolating from one nanostructure (e.g., silver nanowire) to another, sufficient nanostructures must be present in the conductive layer to reach an electrical percolation threshold and become conductive over a specified length or area. The conductivity of the conductive layer is inversely proportional to its resistivity, sometimes referred to as sheet resistance, which can be measured by known methods in the art. For instance, resistivity may be expressed in the forms of ohms/square or ohm/length (e.g., ohm/cm or ohm/m).

The conductivity of the conductive layer correlates to the density of the nanostructures in the mesh. Density refers to the mass of nanostructures per unit area (i.e., surface density) or unit volume. In certain embodiments, e.g., for two-dimensional mesh, the surface density (also referred to as surface loading) may be in the range of 0.05 to 100 μg/cm². In other embodiments, e.g., for three-dimensional mesh, the volume density may be in the range of 0.05 μg-50 mg/cm³.

The conductive mesh is naturally porous, which allows cells and other substances (e.g., a binder or bodily fluid) to access or infiltrate the composite material. In addition, the conductive mesh is flexible and stretchable, especially when hydrated or swollen (e.g., after implantation). Flexibility and stretchability are important features which allow the mesh to be conformal to the body and withstand strain induced by natural body movement and swelling during wound healing.

### Fabrication of Scaffolds

The electrically conductive scaffolds may take any shapes and forms, owing to the combined properties of flexibility and strength of the biocompatible polymers and the conductive mesh. For instance, the scaffolds may be in the forms of threads, films, membranes, tubes, or discs, etc.

In some embodiments, the conductive mesh is at least partially incorporated in the matrix of the biocompatible polymers to form a cohesive or integrated composite material, which is subsequently fabricated into scaffolds.

In other embodiments, a scaffold substrate may be coated on its surface with a coating solution comprising one or more biocompatible polymers and a plurality of conductive nanostructures (e.g., silver nanowires) dispersed in a solvent. Upon drying, an electrically conductive composite material is evenly distributed on the surface of scaffold substrate as a thin film. The scaffold substrate may be made of a biomaterial compatible with or chemically similar to the biocompatible polymers in the coating solution. In certain embodiments, the coated scaffold substrate may be shaped or molded into a scaffold of a desired shape. In other embodiments, the scaffold substrate may have a pre-formed scaffold shape (e.g., a tubular shape) and the thin film of coating solution conforms thereto, resulting in a scaffold with surface conductivity while retaining the pre-formed shape.

### A. Two-Dimensional Scaffolds

Various embodiments provide electrically conductive scaffolds in two-dimensional shapes such as thin membranes. Conductive membranes may be used as an interface between tissues or as a wrap, placed around damaged tissue. Two-dimensional scaffolds may also be rolled or folded into three-dimensional shapes, as described herein.

Thus, in a specific embodiment, a conductive membrane comprises at least one layer of conductive mesh and at least one adjacent layer of biopolymer. In the simplest configuration, a conductive membrane may be formed by sequentially coating a suspension of the conductive nanostructures followed by coating a solution of the biopolymer. Generally speaking, the conductive nanostructure layer may be in the range of about 5-500 nm in thickness, whereas the biocompatible polymer layer may be in the range of about 1-100 μm in thickness. Two or more conductive membranes of the simplest configuration can be laminated together to form thicker, multi-layer conductive membranes.

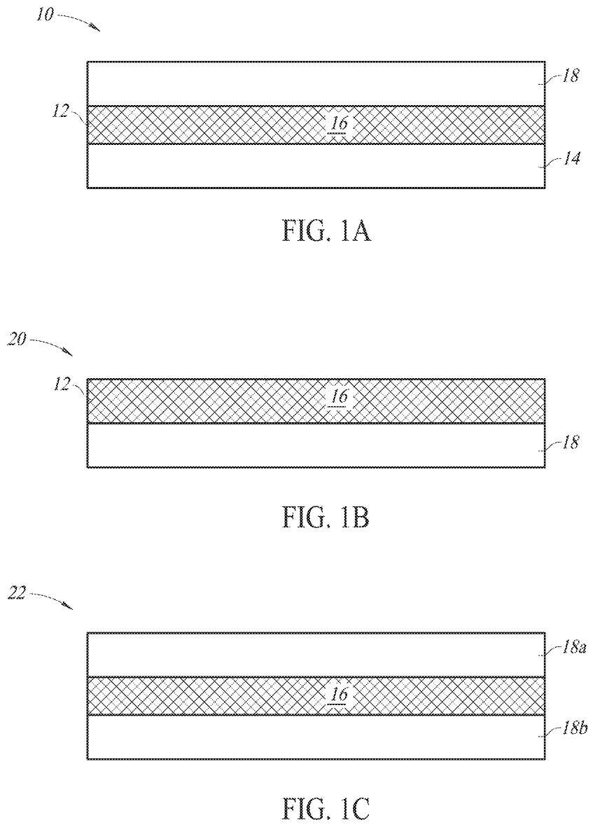

FIGS. 1A-1C show schematically processes for fabricating conductive membranes according to certain embodiments. More specifically, FIG. 1A shows one of the simplest configuration of a conductive membrane (10) formed by coating conductive nanostructures (e.g., silver nanowires) (12) on a plastic substrate (14) to form a nanostructure layer (16), followed by coating a biocompatible polymer layer (18) on top of the nanostructure layer. The substrate may be, for example, polyethylene terephthalate (PET), polycarbonates (PC), polyurethane (PU), cyclic olefin polymers (COP), and the like. The substrate may be coated with a thin layer (e.g., a hydrophobic or hydrophilic coating) to adjust the surface energy as needed. In the illustrated examples, the nanostructure layer may have a surface loading of 0.05 to 100 μg/cm² and the sheet resistance (which depends on the loading density of the nanostructures) is in the range of 1-10,000 ohm/sq. The biocompatible polymer layer is typically 1 micron to 100 microns thick, or more typically, 1 micron to 80 microns thick, or more typically, 1 micron to 60 microns thick, or more typically, 1 micron to 40 microns thick, or more typically, 1 micron to 20 microns thick, or more typically, 1 micron to 10 microns thick.

FIG. 1B shows the simplest configuration of a conductive membrane (20) detached or peeled off from the plastic substrate (14) of FIG. 1A.

FIG. 1C shows a laminated conductive membrane (22) formed by laminating two free standing conductive membranes (20) as shown in FIG. 1B with the respective nanostructure layers facing each other, interposed between first biocompatible polymer layer (18a) and second biocompatible polymer layer (18b).

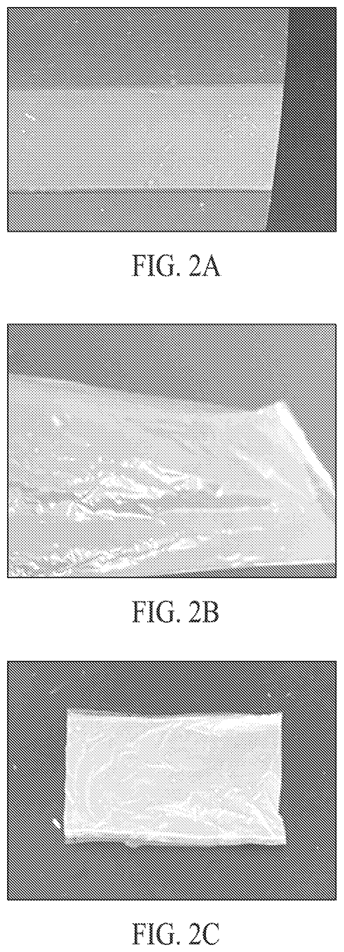

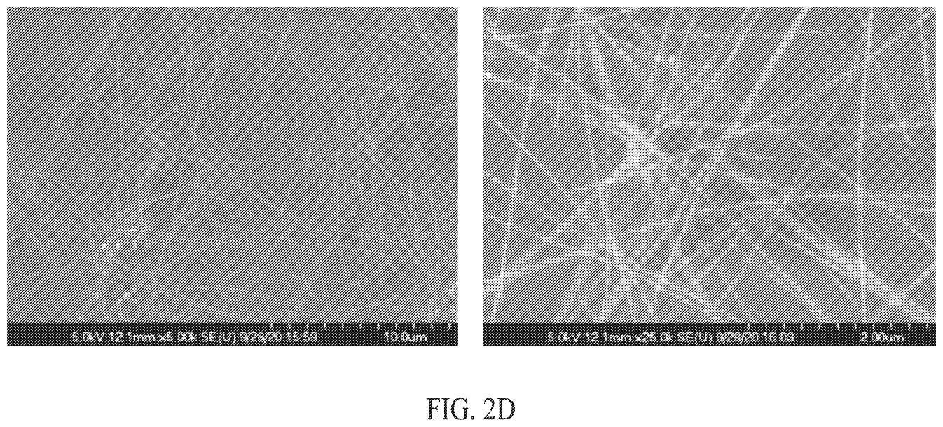

FIGS. 2A-2C are examples of the three configurations as illustrated in FIGS. 1A-1C, respectively, in which a silver nanowire (AgNW) layer is combined with a collagen (e.g., aligned collagen) layer.

FIG. 2D shows the SEM images of the conductive membranes of FIG. 2B (AgNW layer combined with a collagen layer). As shown, the silver nanowires form a mesh structure.

To form the conductive nanostructure layer such as AgNW layer, a coating solution of the conductive nanostructures may be prepared which comprises a suspension of the conductive nanostructures in a solvent such as water, alcohol (e.g., methanol, ethanol, isopropanol, etc.) or a combination thereof. In some embodiments, one or more biodegradable and biocompatible binders are used as coating additives to aid in the formation of a uniform film of conductive nanostructure. The binders allow for a larger coating process window as compared to formulations of nanowires in solvents only. The biodegradable and biocompatible properties of the final composite material are retained. Suitable biodegradable and biocompatible binders include, for example, collagen, gelatin, glycosaminoglycans (GAGs), chitosan, sodium alginate, alginic acid, and synthetic polymers such as polycaprolactone (PCL), polyglycolic acid (PGA), polylactic acid (PLA), poly(lactide-co-glycolide) (PLGA), hydroxypropyl cellulose (HPC), hydroxypropyl methylcellulose (HPMC), and the like.

In preferred embodiments, GAG-based binders are used as a binder or glue between the laminated layers. Examples of GAG include hyaluronic acid, heparan sulfate (heparin), chondroitin sulfate/dermatan sulfate, keratan sulfate.

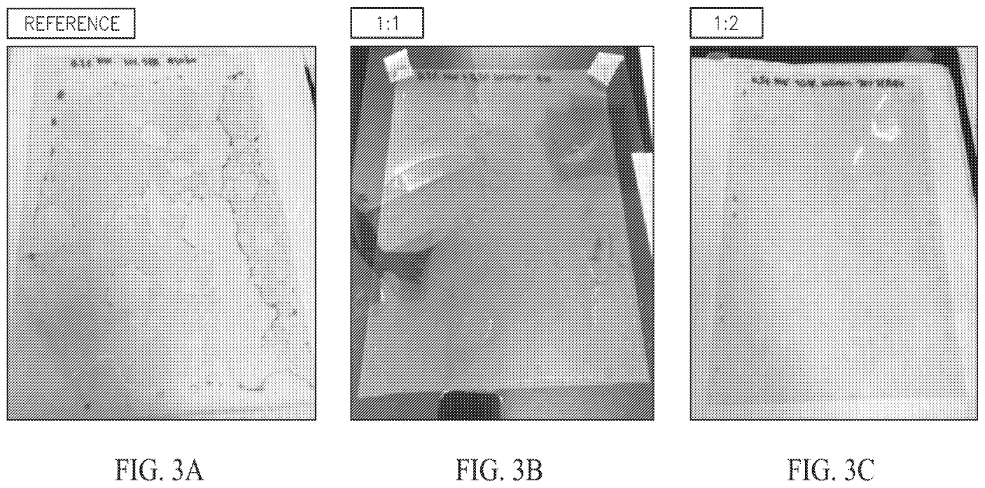

The relative amounts of the conductive nanostructures and the collagen binder can impact the uniformity and continuity of the conductive nanostructure layer, as demonstrated in FIGS. 3A-3C. More specifically, coating solutions of 0.2% AgNW with 0.2% and 0.4% collagen, respectively, were prepared in deionized water (DIW) having 20% isopropanol (IPA). Unless specified otherwise, all percentage amounts or concentrations described herein refer to w/w. As a reference, a binder-less coating solution of 0.2% AgNW in DIW and 20% IPA was also prepared. The coating solutions (including the reference) were coated on PET substrate using Mayer bar coating (#10 bar). After coating, the films were dried at room temperature or elevated temperature (e.g., up to 120° C.) for a sufficient amount of time to fully remove the solvent. Care should be taken to avoid thermal destabilization of the binders.

FIG. 3A shows that, without a binder, the AgNW layer formed lacks uniformity. FIGS. 3B and 3C show the AgNW layers having binders at 1:1 and 1:2 weight ratios respectively. As shown, the collagen binder aids in creating continuous nanowire films.

The AgNW layers are also conductive. Table 1 shows the sheet resistances of the AgNW layers formed from the three coating solutions with increasing relative amounts of collagen binders.

TABLE 1

| AgNW:Collagen Binder | Rs (Ω/□) |
|---|---|
| 1:1 | ~20 |
| 1:2 | ~40 |

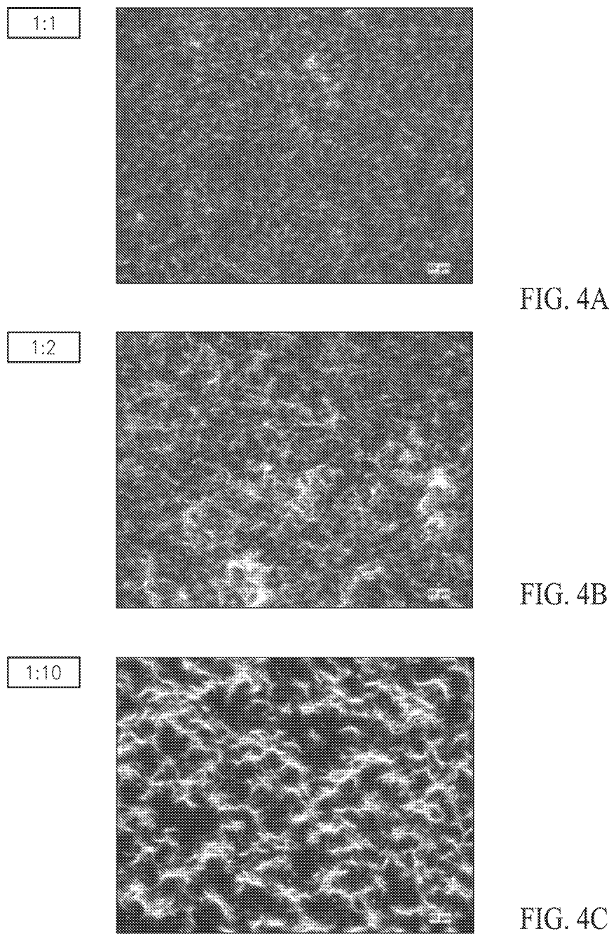

FIGS. 4A-4C are respective dark field images of the collagen/silver nanowire conductive coatings of Table 1. All images were taken at 200×, 10 ms.

Similar to collagen, hyaluronic acid (HA) also aids as a binder for forming a continuous and uniform nanostructure layer. Coating solutions of 0.2% Ag with 0.2% HA and 0.4% HA, respectively, were prepared in a solvent of DIW with 20% IPA. The coating solutions were coated on a PET substrate using Mayer bar coating (#10 bar). After coating, the films were dried at 120° C. for 2 min to fully remove the solvent.

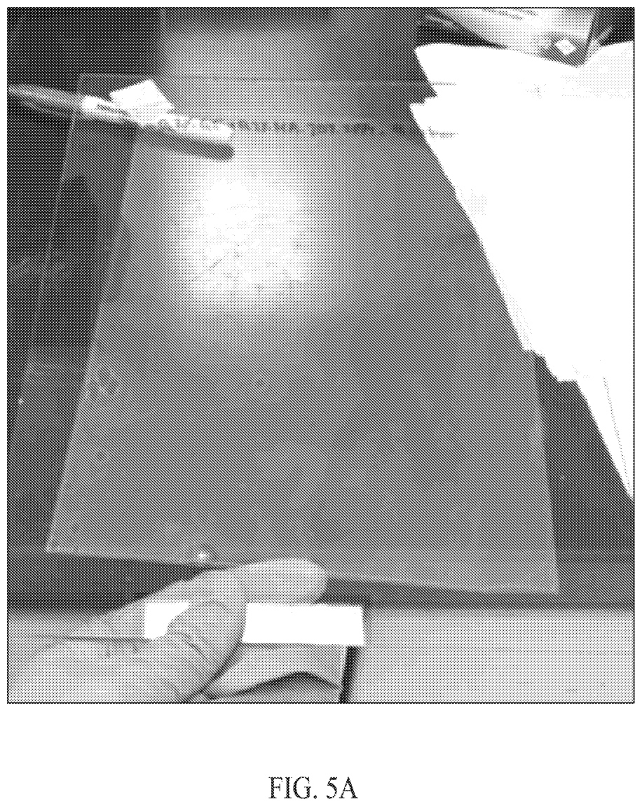

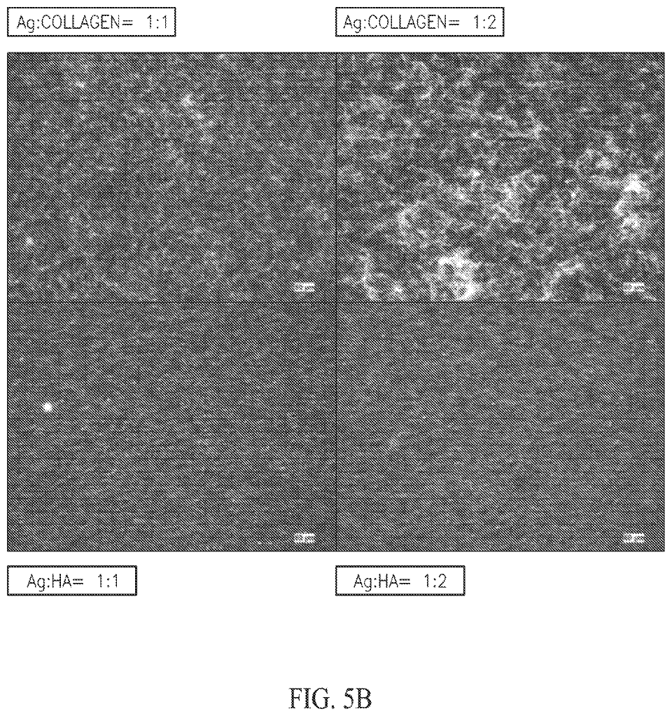

FIG. 5A shows a coated film having a continuous and uniform appearance. The AgNW layers are also conductive, as shown in Table 2.

TABLE 2

| AgNW:HA loading | Rs (Ω/□) |
|---|---|
| 1:1 | ~20 |
| 1:2 | 30.4 |

FIG. 5B shows dark field images of the coated AgNW films on PET substrates using different relative amounts of collagen and hyaluronic acid as binders. All images were taken at 200×, 10 ms.

### B. Three-Dimensional Scaffolds

In some embodiments, the electrically conductive scaffold has a three-dimensional structure, which may or may not have a hollow interior. Tubular scaffolds are particularly suitable for tissue repair by providing a temporary conduit between respective proximal and distal ends of severed or damaged tissue (e.g., severed nerves), allowing the tissue to regenerate within the hollow interior of the scaffold and reestablish connection.

In certain embodiments, a conductive tubular scaffold may be formed by rolling a conductive membrane till the two edges of the membrane meet and overlap to form a cylinder. The cylinder may have a single layer of the conductive membrane or more than one layer for a more structurally sound tube with multiple overlapping wraps of the conductive membrane. The two overlapping edges may be glued together with a biodegradable binder (e.g., heparin), as described herein.

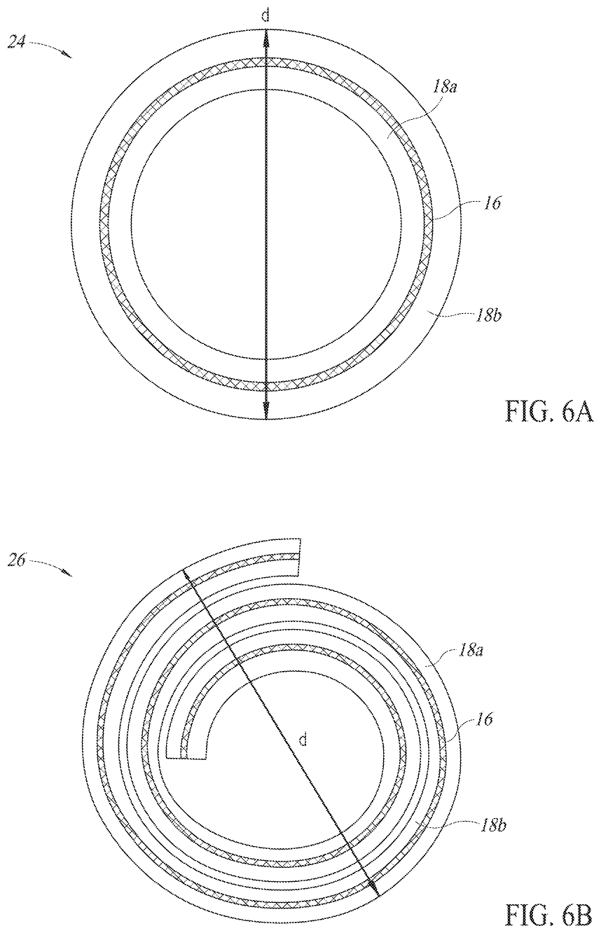

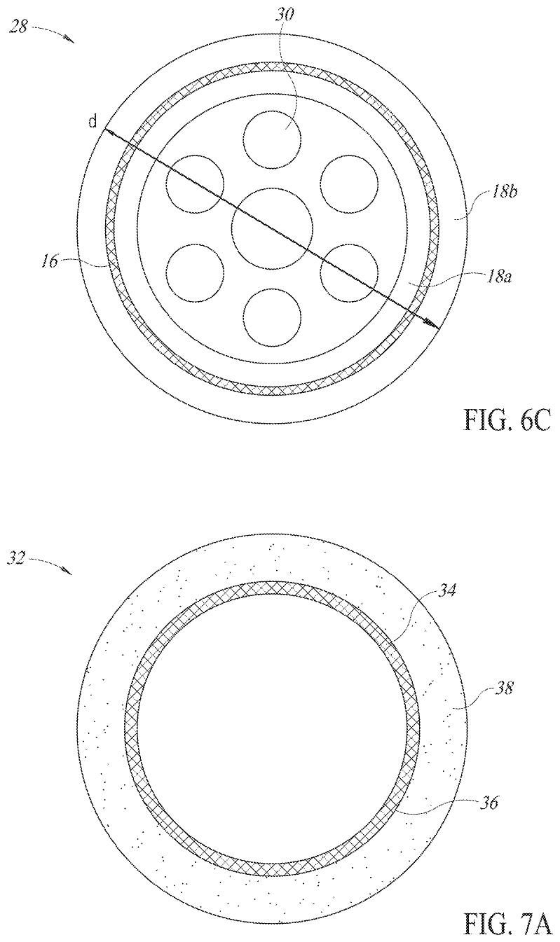

FIG. 6A shows a conductive tube (24) formed by rolling the conductive membrane (22) of FIG. 1C into a single-layer tubular structure. FIG. 6B shows a conductive tube (26) formed by rolling the conductive membrane (22) of FIG. 1C into a multi-layer tubular structure.

The resulting tube of either FIG. 6A or 6B typically has an internal diameter of 1 mm to 10 mm; and a length of 10 mm to 200 mm. The nanostructure layer has a surface loading of 0.05 to 100 μg/cm², having a sheet resistance of 1-10,000 ohm/sq. Each of the biocompatible polymer layer is about 1 micron to 100 microns in thickness.

FIG. 6C shows a conductive tube (28) further containing in its interior space one or more threads (30) of a fibrillar biopolymer. The parallel-oriented threads (30) could further guide the tissue regrowth. The threads can either be conductive or non-conductive and each is about 50-200 μm thick for a conductive tube of 1-10 mm in diameter. Suitable threads may be, for example, the collagen threads marketed under the tradename of BioBridge® (Fibralign Corp.) Alternatively, very thin conductive or non-conductive fibers or ribbons of a biocompatible polymer may be placed within the tubular structure to guide tissue growth. These fibers may be of arbitrary cross section, including hollow structures, with cross-sections of isotropic or anisotropic shapes. At least one cross sectional axis of these fibers is in the range of 1-100 μm in width. By placing these thin conductive or non-conductive threads, fibers, or ribbons inside a thicker, more robust collagen tube, these interior components are capable of serving as an insert to apply electrical stimulation or guide tissue regeneration.

In other embodiments, a conductive tubular scaffold may be formed by coating the interior surface of a tube made of a biopolymer. The tubes may be a preformed collagen tube such as a nerve guide sold under the name of NeuraGen® (Integra LifeSciences), or made of other biocompatible materials such as polycaprolactone (PCL), poly(lactide-co-glycolide) (PLGA), etc.

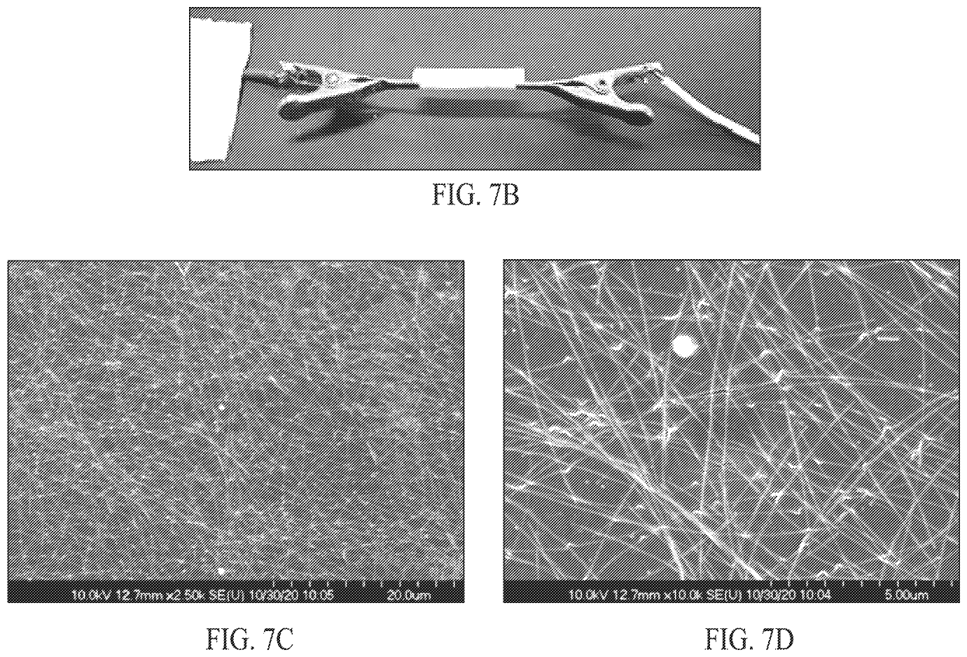

A biopolymer tube (e.g., a collagen tube) can be rendered electrically conductive by coating a conductive mesh on its surface (e.g., the interior surface of the tube). FIG. 7A shows a resulting conductive tube (32) having a uniform conductive layer (34) on the interior surface (36) of a collagen tube (38). The coating may be carried out by incubating the tube interior with a coating solution of conductive nanostructures. For instance, a coating solution of 0.05% silver nanowires (AgNWs) in water or IPA may be prepared, in which a collagen tube (3 mm in diameter and 1 cm in length) is incubated (e.g., dip-coated) to allow a conductive mesh to form in the interior of the pre-formed tubes. The coating can be restricted to the interior of the collagen tube with a protective and removable mask applied on the exterior surface of the collagen tube. The conductive coating can also be formed on the exterior of the collagen tube or on both the interior and exterior of the collagen tube.

The conductive mesh imparts electrical conductivity to the collagen tube, with a resistance in the range of 1-1000 ohm/cm. FIG. 7B shows an end-to-end measurement of the resistance of the conductive tube of about 10 ohm/cm. Resistance can be adjusted by varying the speed with which to withdraw the collagen tube from the same coating solution or by changing the concentration of AgNWs in the coating solution or by varying the number of coating passes. Typically higher AgNW concentration of the coating solution, slower rate of withdrawing the tube from the coating solution and more passes produce coated tubes at lower resistance per length and vice versa.

FIGS. 7C and 7D show SEM images of the interior of the conductive tube under different degrees of magnification. As shown, a mesh of AgNW was formed that confers conductivity.

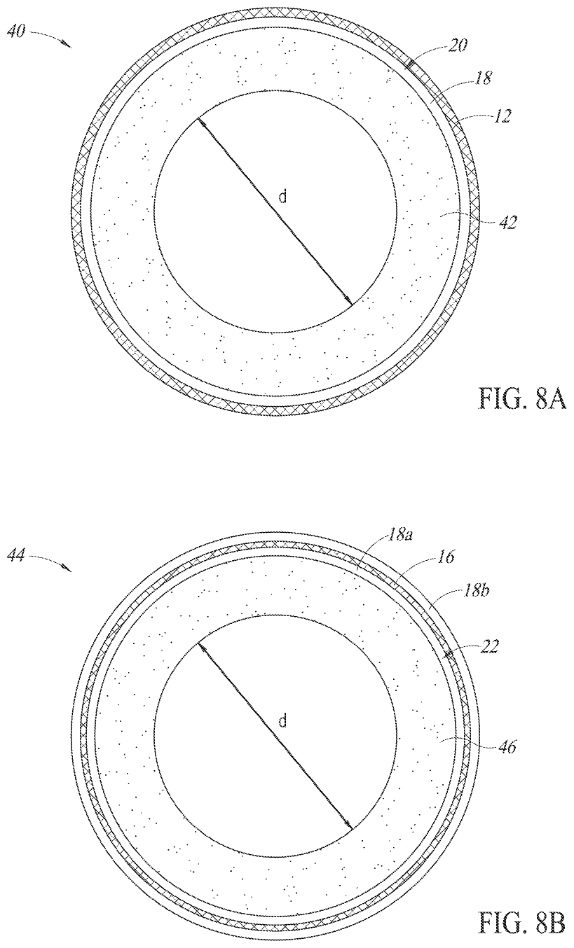

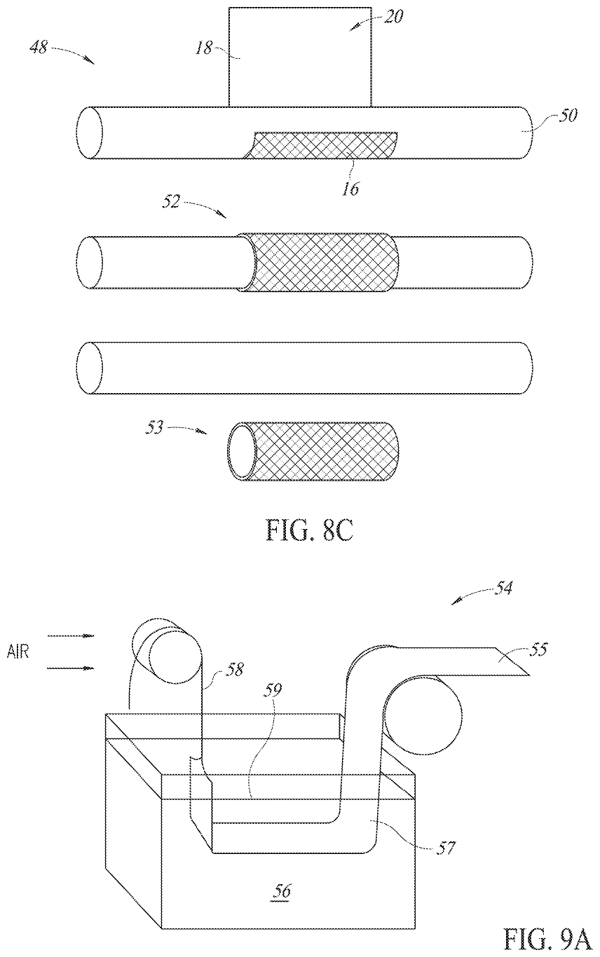

In a further embodiment, a conductive tube may be formed by coating or wrapping a conductive membrane of FIG. 1B or 1C on the exterior of a preformed tube. FIG. 8A shows a conductive tube (40) having a porous collagen tube (42) (<1 mm wall thickness with an interior diameter of 1-10 mm) coated with a conductive membrane (20) of FIG. 1B. FIG. 8B shows a conductive tube (44) having a porous collagen tube (46) (<1 mm wall thickness with an interior diameter of 1-10 mm) coated with a conductive membrane (22) of FIG. 1C. A binder or glue (e.g., heparin) may be used to secure the conductive membrane on the exterior surface of the pre-formed tube. The overall scaffold is electrically conductive.

In yet another embodiment, a conductive tube may be formed by a process (48) shown in FIG. 8C. A conductive membrane (20) (of FIG. 1B) is rolled in a single or multiple layers on a rod (50), which radius determines the interior dimension of the rolled up tube (53). After the removal of the rod (50), a conductive tube or cylinder (53) is formed with the nanostructure layer (16) on the exterior of the tube. A conductive tube having a nanostructure layer on the interior of the tube (not shown) can be similarly formed by reorienting the conductive membrane such the nanostructure layer faces the rod.

### C. One-Dimensional Scaffolds

In some embodiments, the electrically conductive scaffold has a linear, one-dimensional structure of high aspect ratio (e.g., at least 10). The electrically conductive scaffold may be, for example, a filament, a wire, a thread or a suture (collectively “threads”). Linear electrically conductive scaffolds such as sutures are capable of promoting wound healing by providing heat via passage of electrical current (joule heating), or conferring anti-microbial or anti-bacterial properties to the would site. They also find applications as electrodes or wires in implantable medical devices.

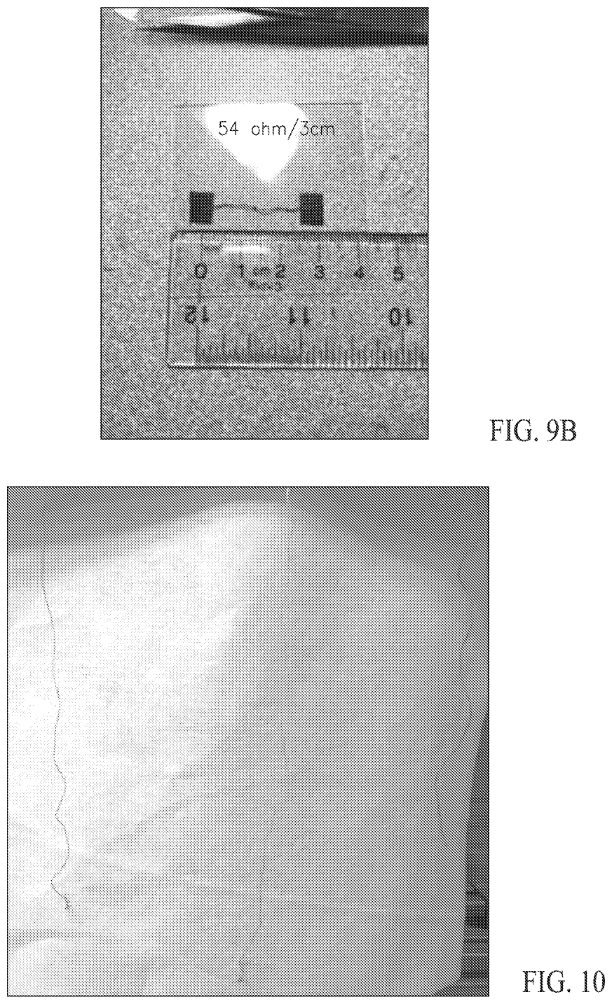

In a specific embodiment, a conductive thread may be fabricated from a conductive membrane having a biocompatible polymer layer and a conductive nanostructure layer (e.g., the structure of FIG. 1B or FIG. 1C). A process (54) is shown in FIG. 9A, wherein a conductive membrane (55), as a dried film, is pulled through a saline solution (56) (e.g., phosphate-buffered saline, PBS) in which the dried film swells and softens into a wet film (57), before being converted into pseudo fibers or thread (58) at the interface (59) of the saline solution and air. More detailed description of forming pseudo fibers may be found in, for example, U.S. Pat. No. 8,513,382.

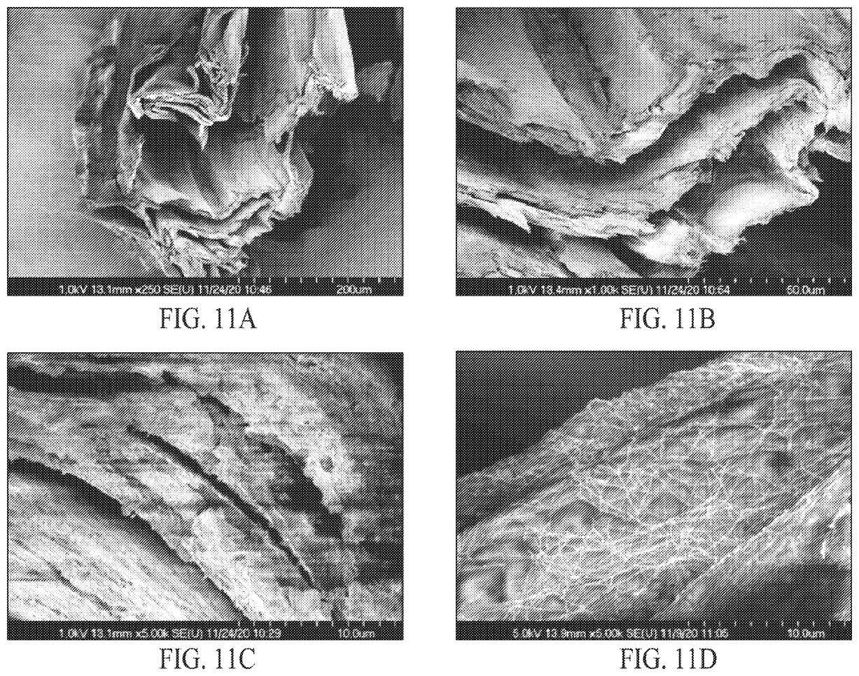

The resulting pseudo fibers are highly conductive, as shown in the end-to-end measurement (54 ohms) of a 3 cm segment (FIG. 9B). The conductivity of the conductive thread can be adjusted by using conductive membranes of different densities of the conductive nanostructures. FIG. 10 shows three conductive threads of different conductivities: 10 ohm/cm (right side), 54 ohm/cm (left side), and 333 ohm/cm (middle).

SEM images of the conductive threads confirm the presence of a nanowires mesh in the conductive thread. FIGS. 11A-11C further show the nanowire mesh interleaved with collagen in a pleated format and FIG. 11D shows silver nanowire network of the thread. Advantageously, porous cross-section promotes fluid transport by capillary forces.

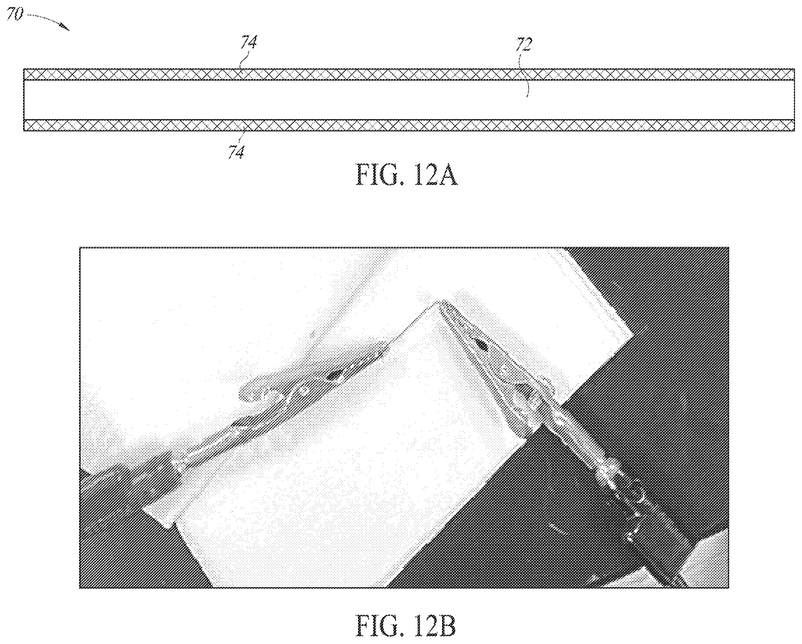

In an alternative embodiment, as shown in FIG. 12A, a conductive thread (70) may be formed by coating a biopolymer thread (72) with a layer of conductive nanostructures (74). The pre-made biopolymer thread may be an industry standard or commercial suture such as catgut sutures, silk, or synthetic (e.g., polyester) sutures. Typically for a conductive suture, the resistance is in the range of 1-1000 ohm/cm by end-to-end measurement. See FIG. 12B.

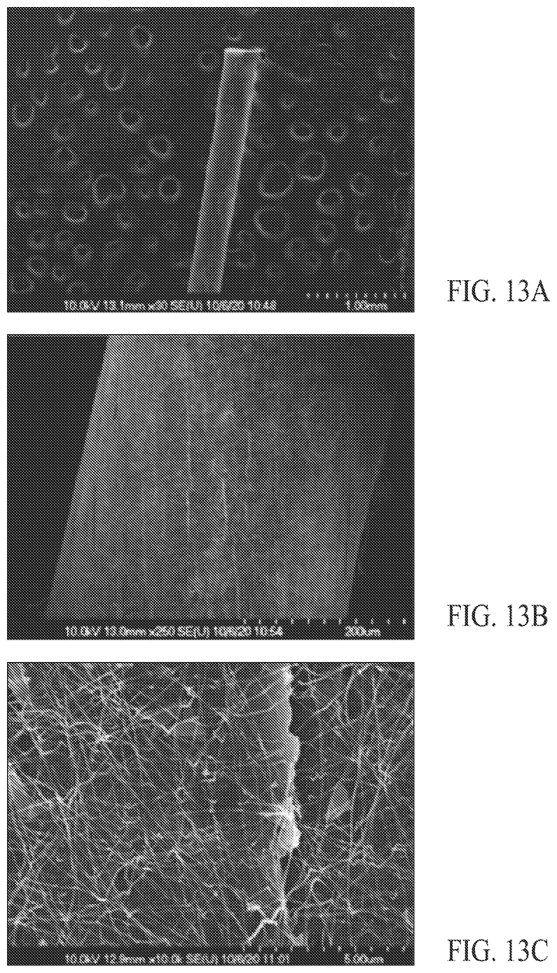

FIGS. 13A-13C show SEM images of AgNW coated catgut sutures in increasing magnifications. As shown in FIG. 13C, a nanowire mesh is present on the exterior of the catgut suture, rendering it conductive.

### Degradation

Collagen-based implants degrade under normal conditions in the body through a process involving phagocytosis of collagen fibrils by fibroblasts, followed by sequential attack by lysosomal enzymes. Thus, the electrically conductive scaffolds can be adapted to fully degrade in vivo following the completion of a treatment (e.g., therapeutic electrical stimulation), thus rendering unnecessary to remove the scaffolds.

The degradation can be slowed by crosslinking collagen, by which intramolecular bonds or intermolecular bonds are formed. Collagen naturally has numerous reactive moieties in the constituent amino acids. For example, reactive moieties such as a primary amine can be covalently coupled to a carboxylic group under suitable conditions to form an amide bond.

Collagen can be crosslinked under physical conditions including, for example, exposing collagen to heat and optionally vacuum, whereby water molecules are removed (i.e., dehydration) while amide bonds are formed.

Collagen can also be crosslinked chemically in the presence of crosslinkers. For instance, zero length crosslinkers such as 1-ethyl-3-(3-(dimethylamino)propyl) carbodiimide (EDC) mediated by N-hydroxysulfosuccinimide sodium salt (sNHS) are capable of conjugating primary amine group and carboxylic group under mild conditions with high crosslinking efficiency.

The degrees of crosslinking are controllable, which further allows control of the scaffold degradation rate.

### Sterilization

The electrically conductive scaffolds may be sterilized by methods described herein. Although conventional sterilization suitable for implantable medical devices can be employed, cares are taken to preserve or minimize any loss of the conductivity. For metallic conductive nanostructures (e.g., AgNW), oxidation or destruction of the network by joule heating caused by a sudden electrical discharge under sterilization conditions may cause a rapid increase in resistance.

In some embodiments, a 22 kGy electron-beam (e-beam) dose may be used. To prevent resistance increase, the conductive scaffolds may be coated with a protective coating, which minimizes any exposure to oxygen. For instance, collagen coating protects the silver nanowire layer during e-beam exposure, resulting in minimal resistance increase. Other top layers such as optically clear adhesives (OCA), or polymer overcoat layers are also effective.

Table 3 shows the resistance shift before and after e-beam sterilization for samples of AgNW conductive films with and without protection. As shown, both collagen coating and OCA prevent loss of conductivity.

TABLE 3

| | | Film | Film | |
|---|---|---|---|---|
| | | resistance | resistance | |
| | | (ohm/sq)/ | (ohm/sq)/ | % Rs |
| sample # | Sample type | before | after | increase |
| 1 | Nanowire only-01 | 12.3 | 18.9 | 54% |
| 2 | Nanowire only-02 | 33.3 | 154.3 | 363% |
| 3 | Nanowire only-03 | 95.5 | 154.3 | 62% |
| 5 | OCA on top of | 11.6 | 11.1 | −4% |
| | nanowire film-01 | | | |
| 6 | OCA on top of | 43.8 | 41.9 | −4% |
| | nanowire film-02 | | | |
| 7 | OCA on top of | 85.8 | 83.7 | −2% |
| | nanowire film-03 | | | |
| 8 | collagen on top of | 487.8 | 497 | 2% |
| | nanowire film-01 | | | |
| 9 | collagen on top of | 55.7 | 43.5 | −22% |
| | nanowire film-02 | | | |
| 13 | overcoat on top of | 53.3 | 42 | −21% |
| | nanowire film-03 | | | |

In other embodiments, the conductive scaffolds may be kept in a container having low air space (e.g., volume of air in the container is less than a critical volume) during sterilization to prevent oxidation or electrical discharge from the ionized atmosphere. In particular, air may be fully evacuated from a container, which is then sealed under vacuum before being subjected to e-beam treatment. Alternatively, inert gas (e.g., Argon (Ar)) may be introduced to prevent resistance increase, allowing for sterilization of conductive scaffolds of any shape, including complex 3D shapes.

Table 4 shows the resistance shift before and after e-beam sterilization for samples of AgNW conductive films exposed to air vs. being sealed in a container filled with argon. As shown, inert air such as argon preserve the film resistance by minimizing oxidation of the AgNW. Samples were measured over 19,999 ohm/sq are indicated as “NC” (non conductive) in the table.

TABLE 4

| | Film | | | Film | |
|---|---|---|---|---|---|
| | resistance | | | resistance | |
| | (ohm/sq)/ | | | (ohm/sq)/ | % |
| Sample | before | orientation | atmosphere | after | change |
| amber | 89 | facing | Ar | 88.4 | −1% |
| bottle-01 | | e-beam | | | |
| amber | 102 | facing | Ar | 136.4 | 34% |
| bottle-02 | | e-beam | | | |
| amber | 123.3 | facing | air | NC | — |
| bottle-03 | | e-beam | | | |
| amber | 127 | facing | air | 6666 | — |
| bottle-04 | | e-beam | | | |
| plastic | 101.6 | facing | Ar | 609.7 | 500% |
| box-01 | | e-beam | | | |
| plastic | 88 | facing | Ar | 343.6 | 290% |
| box-02 | | e-beam | | | |
| plastic | 90.3 | facing | air | NC | — |
| box-03 | | e-beam | | | |
| plastic | 80.5 | facing | air | NC | — |
| box-04 | | e-beam | | | |
| centrifuge | 90.9 | facing | Ar | 104.1 | 15% |
| tube-01 | | e-beam | | | |
| centrifuge | 97.2 | facing | Ar | 124 | 28% |
| tube-02 | | e-beam | | | |
| centrifuge | 90.3 | facing | air | 6000 | — |
| tube-03 | | e-beam | | | |
| centrifuge | 90.1 | facing | air | NC | — |
| tube-04 | | e-beam | | | |
| glass | 108.9 | facing | Ar | 310.5 | 185% |
| bottle-01 | | e-beam | | | |
| glass | 90.9 | facing | air | NC | — |
| bottle-02 | | e-beam | | | |

Similarly, conductive films maintained in containers with minimal surrounding air or alternatively fully evacuated by vacuum can also withstand the sterilization process without losing conductivity. Table 5 demonstrates that minimal changes in resistance are observed after e-beam exposure for samples of bare AgNW conductive films kept in plastic envelops with low air (e.g., envelops flattened to push air out).

TABLE 5

| | Film | | | Film | |
|---|---|---|---|---|---|
| | resistance | | | resistance | |
| | (ohm/sq)/ | | | (ohm/sq)/ | % |
| Sample | before | orientation | atmosphere | after | change |
| plastic | 104.8 | facing | air | 101.7 | −3% |
| envelop-01 | | e-beam | | | |
| plastic | 107.9 | facing | air | 104.8 | −3% |
| envelop-02 | | e-beam | | | |
| plastic | 104.8 | facing | air | 105.3 | 0% |
| envelop-03 | | e-beam | | | |
| plastic | 113.1 | facing | air | 110.2 | −3% |
| envelop-04 | | e-beam | | | |
| plastic | 123.4 | lay flat | air | 121.9 | −1% |
| envelop-05 | | | | | |
| plastic | 110.9 | lay flat | air | 109.1 | −2% |
| envelop-06 | | | | | |

In other embodiments, it is helpful to stabilize nanowire film and minimize resistance increases by packaging the conductive scaffolds in anti-static bags or conductive bags, which are capable of dissipating charges. This packaging method can be further combined with minimizing the air headspace, utilizing inert gas (Ar), or removing air via vacuum packaging. Table 6 shows that there is little resistance shift before and after e-beam sterilization for samples of bare AgNW conductive films kept in a conductive bag or anti-static bag.

TABLE 6

| | Film resistance | Film resistance | % |
|---|---|---|---|
| | (ohm/sq) | (ohm/sq) | change |
| packing | before | after | Rs |
| conductive bag-01 | 89.9 | 89.7 | −0.22% |
| conductive bag-02 | 101 | 98.9 | −2.08% |
| anti-static bag -01 | 100.5 | 88.7 | −11.74% |
| anti-static bag -02 | 79.5 | 73.4 | −7.67% |

In other embodiments, air may be fully evacuated from a container, which is then sealed under vacuum before being subjected to e-beam treatment. It is known that e-beam sterilization in air can produce ozone which can degrade the conductivity of the AgNW conducting films. Removing air allows the e-beam process to take place with no ozone formation and thus no damage to the AgNW. Table 7 below shows the resistance shift before and after e-beam sterilization for samples of bare AgNW conductive films ⅛″ strips sealed in glass ampoules under vacuum.

TABLE 7

| | Film resistance | Film resistance | % |
|---|---|---|---|
| | (ohm) | (ohm) | change |
| Packing | before | after | R |
| Sealed under vacuum-01 | 773 | 805 | 4% |
| Sealed under vacuum-02 | 517 | 509 | −2% |
| Sealed under vacuum-03 | 541 | 553 | 2% |

### Patterning of Conductive Scaffolds

The conductive scaffolds described herein can be patterned by various methods. For example, the metal nanowire layer can be patterned by traditional lithography methods with a mask to define a pattern and the exposed areas can be removed by physical wiping or chemical etching before coating the biocompatible polymer. Another method is to mask the metal nanowire layer/biopolymer composite to define a pattern and then exposed areas can be removed by dissolving the biocompatible polymers. Alternatively, the metal nanowire layer/biopolymer composite can also be cut into certain dimensions and shapes based on a predefined pattern depending on application. Laser ablation is another suitable method for making a pattern in the conductive nanostructures or a conductive scaffold. Alternatively, a nanowire suspension may be printed on a substrate to generate a pattern by various printing techniques including inkjet, flexo, gravure, screen, or other methods.

### Use of Conductive Scaffolds

The conductive scaffolds described herein are capable of stimulating and promoting tissue regeneration, including nerve repair. In other embodiments, they may act as conductive scaffoldings to facilitate electrical stimulation to patients with stroke, Alzheimer's, or glioblastoma. In various embodiments, the electrically conductive scaffolds may also be implanted as neural tissue interface, drug release depot, or as image contrast agents due to their radio opacity. In further embodiments, they may also be used as cell transfer scaffolds with in-vitro preconditioning for stroke, wound healing, myocardial infarction, etc.

### A. Electrical Contacts

For use in electrostimulation, the conductive scaffolds are provided with electrical contacts for making the electrical connection to a power source. Typically, biocompatible, and inert components (e.g., titanium or gold) maybe used to form the electrical contacts.

One embodiment provides a bundle (60) of conductive threads (62) by cutting or sizing the threads based on the length of the implanted site. The number of threads is based on the width of the implanted site and the radial dimension of the threads. Typically, three threads may cover 2 mm in width. The threads (62) are then arranged in parallel.

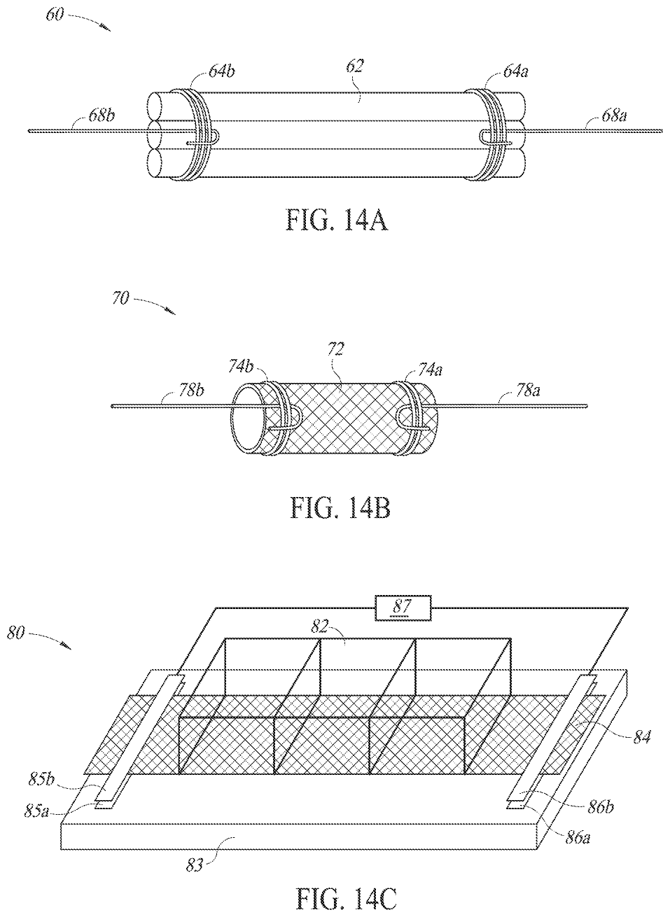

To prepare the electrical contacts, remove the insulation at respective ends (5 mm) of a 2-3 cm long insulted gold wire or other conductive metal wires, form a U shape at the exposed ends. Place the U-shape part on top of the bundle (60) of threads (62) at two respective ends, followed by clipping the gold wires (68a and 68b) and the threads with medical grade Titanium microclips (64a, 64b). See FIG. 14A.

Another embodiment provides a conductive cylinder prepared according to a process as shown in FIG. 8C. More specifically, a rod maybe selected with a predetermined diameter based on the width of the implanted site. Typically, a rod with a diameter of 1.3 mm results in a conductive cylinder of about 2 mm in width once flattened.

To prepare the electrical contacts, remove the insulation at respective ends (5 mm) of a 2-3 cm long insulted gold wire or other conductive metal wires, form a U shape at the exposed ends. FIG. 14B shows a conductive cylinder (70) having nanostructures on the exterior (see also FIG. 8C), wherein gold wires (78a and 78b) and the cylinder (70) are clipped together with medical grade Titanium microclips (74a, 74b).

To test electrical stimulation in vitro on cell behavior, a culture system (80) as shown in FIG. 14C may be used. The system is suitable for use with upright and inverted microscopes. More specifically, a 3-well chamber (82) may be used for cell culture and immunofluorescence staining. A conductive collagen membrane (84), with the conductive nanowires (e.g., silver nanowires) facing down, is placed between the chamber (82) and a glass bottom (83). At either end of the chamber, a first pair of conductive tapes (85a, 85b) and a second pair of conductive tapes (86a, 86b) secure the conductive collagen membrane (84). The bottom conductive tapes (85a, 86a) provide electrical contacts with AgNW/collagen membrane. The conductive tapes may be copper tapes, or conductive metal thin films such as gold sputtered thin film on the glass bottom (83). The top conductive (e.g., copper) tapes (85b, 86b) secure the membrane (84) down and connected with respective bottom conductive tapes (85a, 86a) to provide electrical connection to electrical stimulation device (87).

Resistance in cell culture can be monitored during electrical stimulation. The conductive scaffold in the in vitro well chamber maintained conductivity during the cell culture process, as shown in Table 8 below.

TABLE 8

| | Resistance |
|---|---|
| Processing Steps | Change % |
| as made in vitro well chamber | — |
| wet the sample in PBS for 5 min | 9% |
| sterilize the sample in the ethanol (70%) for 10 min | −4% |
| rinse the sample in PBS for 10 min | −2% |
| rinse the sample in PBS for 10 min | −9% |
| add cell media DMEM with 10% FBS and incubate at 37° C. | −13% |
| for 30 min | |
| seed HFB cells | −13% |
| culture cells at 37° C. for 1 day | 42% |
| apply electrical stimulation at 37° C. for 1 hour | 42% |

PBS buffer: phosphate buffered saline buffer;
DMEM media: Dulbecco's Modified Eagle Medium;
FBS: Fetal Bovine Serum
HFB cell: human fibroblast cell
Electrical stimulation: square wave, 100 Hz, 100 mV, for 1 hour.

### B. Nerve Repair

In preferred embodiments, a tubular conductive scaffold is provided as a conduit for restoring or repairing damaged or severed nerves. Conventionally, it is believed that five different phases of nerve regeneration take place inside a hollow conduit (e.g., collagen tube). The phase corresponds to the sequenced phases of Wallerian degeneration and resulting regeneration mechanism. Phase I corresponds to the fluid phase, where the conduit is filled with plasma exudate containing neurotrophic factors and ECM molecules. This phase takes place a few hours after injury. Phase II corresponds to the matrix formation, where fibrin cables are formed along the gap around 1 week after injury. Phase III is the cellular phase, where Schwann cells invade the gap, migrate and proliferate. They tend to align along the fibrin cable, forming the Bands of Bungner. Phase IV is axonal phase, which occurs around 2 weeks after injury. The re-growing immature axons use the biological cues provided by Schwann cells to reach their distal targets. Phase V corresponds to the myelin phase. At this time, around 3 weeks post-injury, Schwann cells shift to a myelinating phenotype and produce myelin which is wrapped around each axon, forming the mature myelinated axons. See, e.g., Bioeng. Biotechnol., 22 Nov. 2019|Volume 7|Article 337.

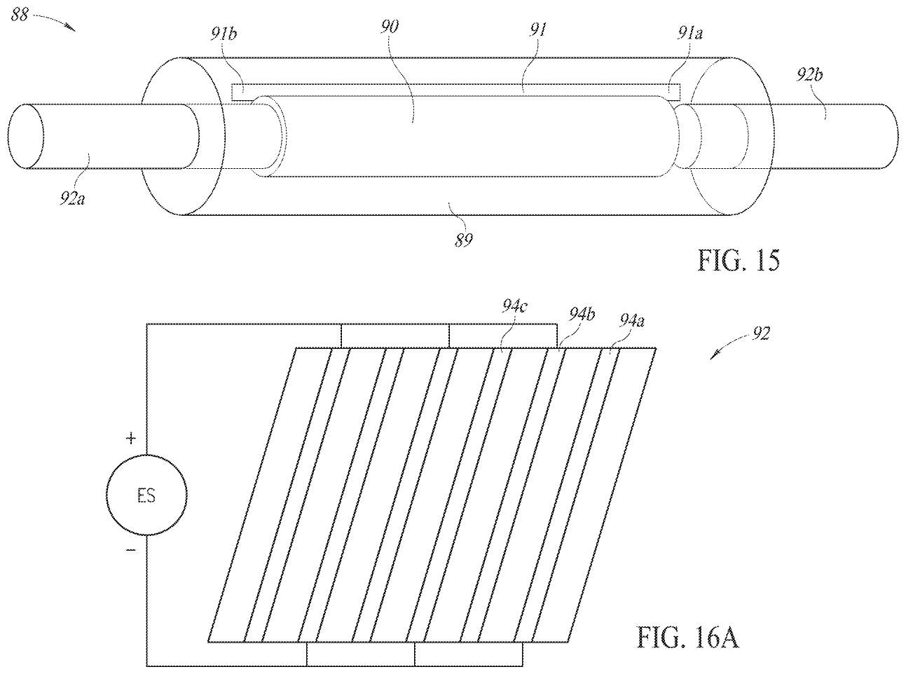

The conductive tubular scaffolds according to embodiments of this disclosure facilitate nerve tissue growth with or even without electrical stimulation. FIG. 15 shows such a tubular conductive scaffold (88), made of a collagen tube (89) having, in its interior, a conductive insert (90) and a conductive strip (91). In particular, the conductive strip has respective contact flaps (91a, 91b) at both ends of the scaffold to establish electrical contacts with the proximal end (92a) and distal end (92b) of a severed nerve.

### C. Wound Treatment

In preferred embodiments, a conductive bandage made of conductive membrane scaffold is provided for repairing damaged skin, cutaneous wounds. Cutaneous wounds generate endogenous electric currents (the “current of injury”) which are involved in numerous processes of wound healing. Electrical stimulation (ES) may promote chronic wound healing by imitating the natural electrical current that occurs in cutaneous wounds. ES affects all the stages of wound healing and has been the most studied biophysical device for healing chronic wounds to date. See, e.g., Experimental Dermatology, vol. 26, no. 2, pp. 171-178, February 2017.

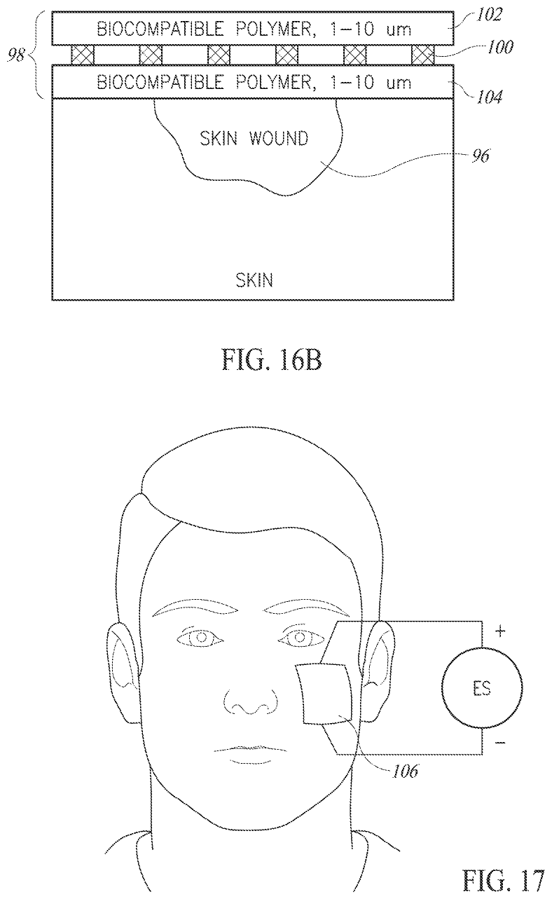

FIG. 16A shows a conductive dressing/bandage (92) made of the collagen/AgNW/collagen membrane. Because of collagen's chemotactic properties on wound fibroblasts, the collagen layer stimulates cellular migration and contributes to new tissue development by creating an environment that fosters healing. The AgNW layer is patterned to regions of conductive lines (94a, 94b, 94c, ect.) based on the specifics of the wound geometry and can be used to provide electrical stimulation.

The conductive bandage covers the entire wound and can provide ES to heal the wound at a constant rate. FIG. 16B shows schematically an open skin wound (96) is covered by a conductive bandage (98) having a patterned nanostructure layer (100) interposed between two biocompatible polymer layers (102, 104).

### D. Cosmetic Treatment

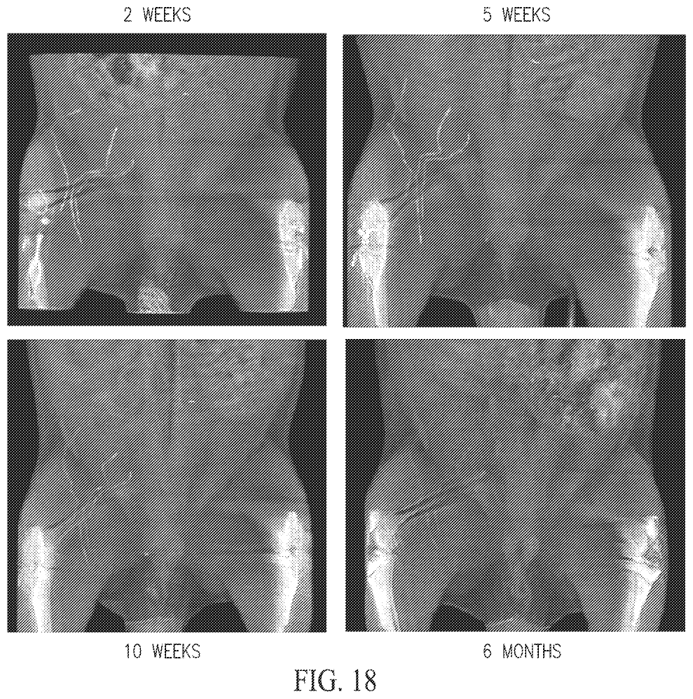

In preferred embodiments, a thin transparent bandage made of a conductive membrane scaffold is provided for repairing wounds or scars on a face or other exposed areas. FIG. 17 shows a transparent conductive dressing/bandage (106) made of the collagen/AgNW/collagen membrane applied to facial skin. The collagen layer stimulates cellular migration and contributes to tissue regeneration by creating an environment that fosters healing. The AgNW layer is patterned into regions of conductive lines based on specifics of the wound areas and can provide ES. The thin collagen layer and the conductive AgNW layer can be transparent which is in some cases has a preferred aesthetic.

### E. In Vivo Visualization

Scaffolds formed with conductive nanostructures are readily detectable or visualized by conventional imaging techniques such as MM and CT because the conductive nanostructures could serve as contrast agents. FIG. 18 shows the CT images taken 2 weeks, 5 weeks, 10 weeks, and 6 months after implanting conductive threads made of aligned collagen and AgNW mesh. As shown, the conductive threads are visible in all images; however, 10 weeks following the implantation, the image of the conductive threads became markedly weaker, indicating significant in vivo degradation of the conductive thread. The degradation rate of the conductive threads can be tuned by the degree of collagen crosslinking, the loading of the AgNW, and the diameter of the AgNW.

### EXAMPLES

### Example 1

### Crosslinking and Degradation

Conductive scaffolds of silver nanowires (AgNW)/collagen membranes (FIG. 2B) were crosslinked (physically or chemically) followed by degradation test.

To physically crosslink collagen, dehydrothermal (DHT) crosslinking was conducted in a chamber under vacuum (28˜30 In·Hg), and temperature in the range of 90-110° C. for a duration 24-72 hrs. It should be noted that the degree of crosslinking is controllable by adjusting the temperature and duration. The degree of crosslinking in turn influences the rate of degradation.

In vitro enzymatic degradation test of the AgNW/collagen membrane scaffolds was performed by incubation in bacterial collagenase (100 U/ml) for 24 h. The degraded collagen in solution was quantified by reacting with 2% ninhydrin and measuring Absorbance (Abs) at 570 nm. The degradation level of a non-crosslinked control sample was set at 100%.

Table 9 shows that the degradation of DHT-crosslinked scaffolds slowed as compared to the non-crosslinked control scaffold.

TABLE 9

| Sample | Abs, normalized | % of Control |
|---|---|---|
| Non-crosslinked, control | 1.009649 | 100.0% |
| DHT cross-linked for 48 hrs | 0.859341 | 85.1% |

To chemically crosslink the scaffolds, crosslinking agents 1-ethyl-3-(3-dimethyl aminopropyl)-1-carbodiimide hydrochloride (EDC, 0.2 mg/ml) and N-hydroxysulfosuccinimide sodium salt (sNHS, 0.22 mg/ml) were used, followed by 4 washes in phosphate-buffered saline (PBS) and 2 washed in deionized water. It should be noted that the degree of crosslinking is controllable by adjusting concentrations of EDC and sNHS. The degree of crosslinking in turn influences the rate of degradation.

Table 10 shows that the degradation of chemically crosslinked scaffolds slowed as compared to the non-crosslinked control scaffold

TABLE 10

| Sample | Abs, normalized | % of Control |
|---|---|---|
| Non-crosslinked, control | 1.442356 | 100.0% |
| Chemical crosslinking | 1.204082 | 83.5% |

### Example 2

### Nerve Repair—Animal Model

Conductive nerve guidance conduits (NeuraGuide™ devices) were produced and evaluated pre-clinically using a rat sciatic nerve repair model at the Washington University Medical School in St. Louis. The NeuraGuide™ device group (n=10) was coupled with therapeutic electrical stimulation across the nerve gap and through the device during the first week of the recovery period. Details of the wireless electrical stimulation system used in this study can be found in J Neurosurg MacEwan et al., 130:486-496 (2019). Contrasted with the previous work, the two electrical leads from the wireless stimulator each terminated at a cuff electrode that were fitted to the proximal and distal nerve stumps to allow electrical stimulation across the nerve gap through the conductive NeuraGuide™ device. A positive control group (n=6) was included for comparison purposes, using an industry standard collagen nerve guidance conduit (NeuraGen® Nerve Guide, Integra Lifesciences). Electrophysiology measurements were performed to assess functional recovery at 12-weeks post-surgery, and histological analysis was performed at 18 weeks post-surgery to evaluate axonal regeneration and biocompatibility.

### Device Design

A NeuraGuide™ nerve guidance conduit was designed to bridge a transected nerve and promote axonal regeneration across the gap. Topological, electrical, and biochemical cues that act together to stimulate nerve growth and repair the transected nerve are integrated into the device.

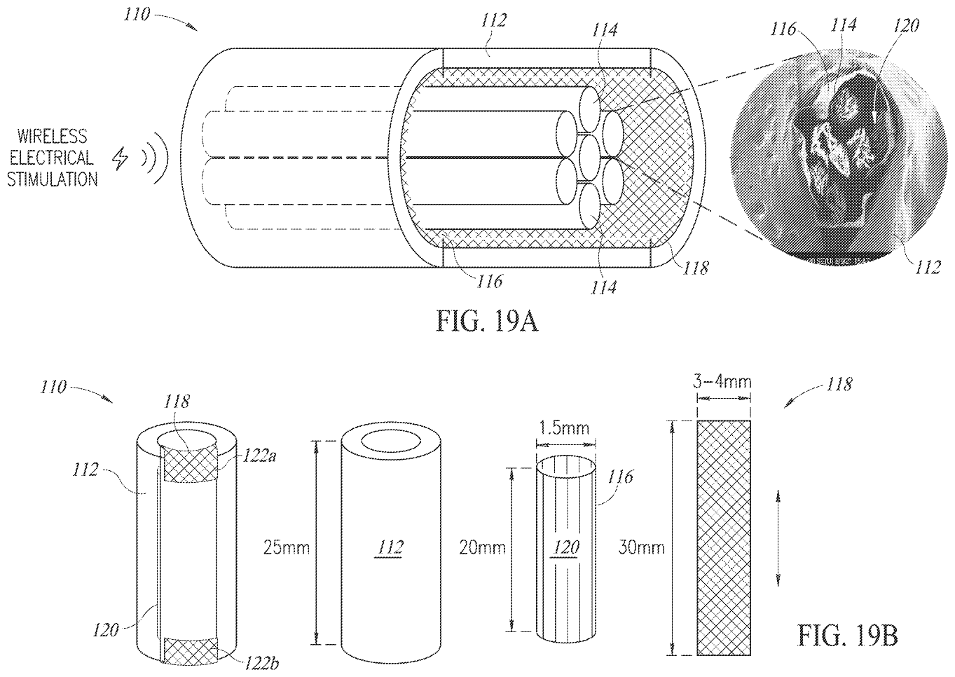

FIG. 19A illustrates the architecture of the NeuraGuide™ device (110), noting that many variations in device design are possible, while preserving the key features of an electrically conductive scaffold that integrates a precisely aligned collagen matrix with a conductive nanowire network to promote nerve repair and enable the application of therapeutic electrical stimulation.

The NeuraGuide™ device (110) comprises a microporous outer collagen tube (112) to isolate and protect the transected nerve (not shown), as well as to provide macroscopic guidance for nerve growth. The outer collagen tube (112) may be the industry standard NeuraGen® Nerve Guide (Integra LifeSciences). This outer collagen tube has sufficient mechanical strength to allow for suture attachment to the proximal and distal nerve stumps. The interior of the collagen tube contains collagen fibers (114) of 50-300 microns in diameter (Biobridge®), which have a highly porous cross section to promote capillary flow, and an aligned fibrillar surface nanostructure. The collagen fibers (114) extend within the interior to provide topological cues to guide axonal growth across the gap between the nerve stumps, and biochemical cues arising from its native collagen structure that presents ligands recognized by integrin receptors. The collagen fibers may be collected into a bundle to by wrapping them with a thin collagen membrane (116) where the collagen fibrils are also oriented along the direction of the nerve gap. These fibers wrapped by the thin collagen membrane (116) are collectively referred to as the “insert” (120) and placed within the collagen tube, leaving a few millimeters of open space at either end where the nerve stumps will be inserted. Finally, a thin conductive collagen membrane strip (118) containing a conductive network of silver nanowires is located at the interior tube wall, spanning the entire length of the tube.

The construction of the NeuraGuide™ device is alternately illustrated in FIG. 19B. As shown, the ends (122a, 122b) of the thin conductive collagen membrane strip (118) exit the porous tube (112) and are folded back and tacked to the exterior of the tube using a drop of liquid collagen solution as an adhesive. The two ends of the conductive strip make physical contact with the proximal and distal nerve stumps, and provide a continuous electrical connection across the nerve gap.

### Sterilization

NeuraGuide™ nerve guidance conduits were individually placed in metallized bags. Care was taken to minimize the air space within the bag. Devices were e-beam sterilized using 2-pass procedure to deliver a total dose of 22 kGy.

### Animal Model

Rat Nerve Repair Model: Animals (male Lewis rats, 250-300 g) had undergone nerve transection/interpositional nerve graft repair of the right sciatic nerve with the NeuraGuide™ NGC and NeuraGen® conduit as positive control. NeuraGen® conduits are generally recognized as a standard in nerve repair surgery. The graft repair were followed by 6 days of electrical stimulation for the NeuraGuide™ treatment group, and post-operative observation for 6 weeks, 12 weeks, with functional recovery assessed through electrophysiological measurements. At 18 weeks, axonal regeneration and biocompatibility were evaluated via gross observation and histological analysis of explanted sciatic nerve samples.

Surgical Procedure: Nerve Transection/Interpositional Nerve Repair: Animals had been anesthetized using 4% Isoflurane/96% oxygen (induction) and 2% Isoflurane/98% oxygen (maintenance) administered by inhalation. Following preparation and sterilization of the skin, the right rat sciatic nerve were exposed through a muscle-splitting incision followed by blunt dissection. All microsurgical procedures were performed under operating microscope. The sciatic nerve were transected with fine iris scissors, then repaired with either a 24 mm NeuraGuide™ NGC or a 24 mm NeuraGen® nerve conduit by suturing to the proximal and distal nerve stumps using four 10-0 nylon sutures (Sharpoint). As a result, recipient nerves in all groups had consistent nerve gaps of 20 mm. After implantation, the incision was irrigated, and the muscle fascia and skin were closed in two layers using 5-0 polyglactin (Vicryl) and 4-0 nylon suture (Ethilon), respectively. Animals were closely monitored prior to returning to the central housing facility.

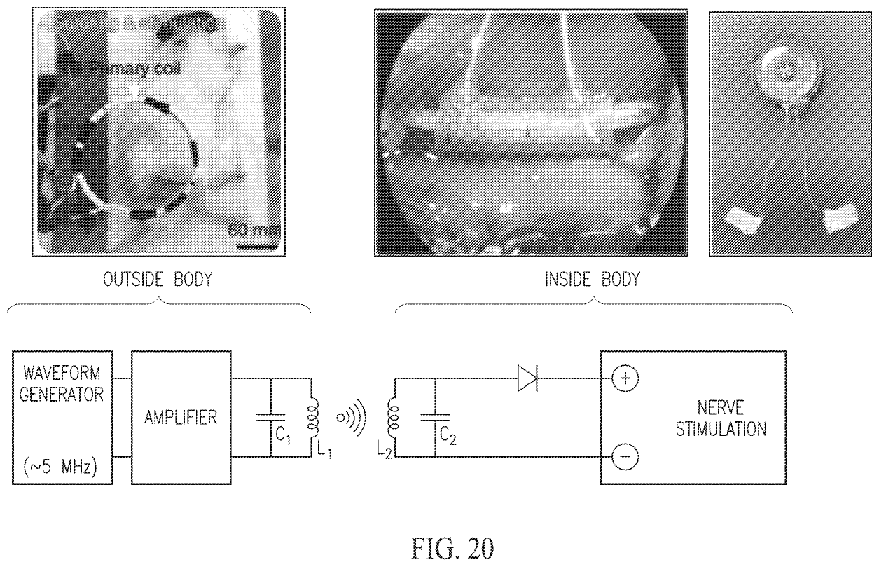

Surgical Procedure: Implantation of Wireless Nerve Stimulator. Following the nerve repair, one wireless nerve stimulator was implanted into each animal treated with the NeuraGuide™ device. Blunt dissection was utilized to create a subcutaneous pocket extending 5 cm from the site of the nerve injury. A transient implantable nerve stimulator was then implanted into the subcutaneous pocket and resorbable leads and nerve cuff were routed to the exposed rat sciatic nerve. The integrated cuff electrodes were then microsurgically fitted to the injured nerve both proximal and distal to the nerve transection, to enable electrical stimulation from proximal nerve stump through the NeuraGuide™ device bridging the nerve gap, to the distal nerve stump. Immediately post-operatively, a wireless transmitter coil was placed over each animal and centered over the implanted nerve stimulator. See FIG. 20. A 5 MHz carrier frequency was utilized to wirelessly power the implanted transient stimulator. Modulation of the carrier frequency generated cathodic, monophasic electrical impulses in implanted wireless receivers (duration=200 usec, amplitude=3.0 V, frequency=variable, timing=variable) which were applied to the rat sciatic nerve across the nerve gap. Implanted nerve stimulators thereby delivered brief and repeated electrical stimulation to injured rat sciatic nerves. Specifically, implants were adapted to electrically stimulate interfaced rat sciatic nerve tissue for periods of 1 hour a day for 6 consecutive days at frequencies of 20 Hz. Following electrical stimulation, the animals recovered and were allowed to return to their enclosure.

Measurement of Nerve Conduction/Electromyography: Rat sciatic nerve function were assessed in situ by examining compound neural action potential (CNAP) propagation, and electromyography (EMG). Cathodic, monophasic electrical impulses (duration=50 usec, frequency=single, amplitude=0-3 mA) were generated by an isolated pulse stimulator (Model 2100, A-M Systems Inc.) and delivered to the rat sciatic nerve proximal repair site via epineural hook electrodes. Resulting CNAPs and EMGs were then differentially recorded distal to the repair site using bipolar silver microwire electrodes (4 mil, California Fine Wire). Measured signals were band-pass filtered (LP=1 Hz, HP=5 kHz, notch=60 Hz) and amplified (gain=1000×) using a two-channel microelectrode AC amplifier (Model 1800, A-M Systems Inc.) before being recorded on a desktop PC equipped with a data acquisition board and custom Matlab. Stimulus amplitudes were incrementally increased to determine stimulus threshold and maximal peak-to-peak amplitude of evoked CNAP and EMG responses.

Histomorphometric Evaluation of Regenerated Nerve Tissue: Samples of explanted nerve tissue were fixed in 3% gluteraldehyde in 0.1 M phosphate buffer (pH=7.2), post-fixed with 1% osmium tetroxide, ethanol dehydrated, and embedded in Araldite 502 epoxy resin (Polysciences). For each sample, cross sections <1 um thick were cut, stained with 1% toluidine blue and examined using light microscopy and evaluated for overall nerve architecture, quantity of regenerated nerve fibers, degree of myelination, and Wallerian degeneration. Quantitative analysis was performed using a semi-automated digital image analysis system linked to a custom software package adapted for nerve morphometry. The following morphometric indices were calculated using primary measurements: number of nerve fibers, nerve fiber density (fiber number/mm²).

### Results

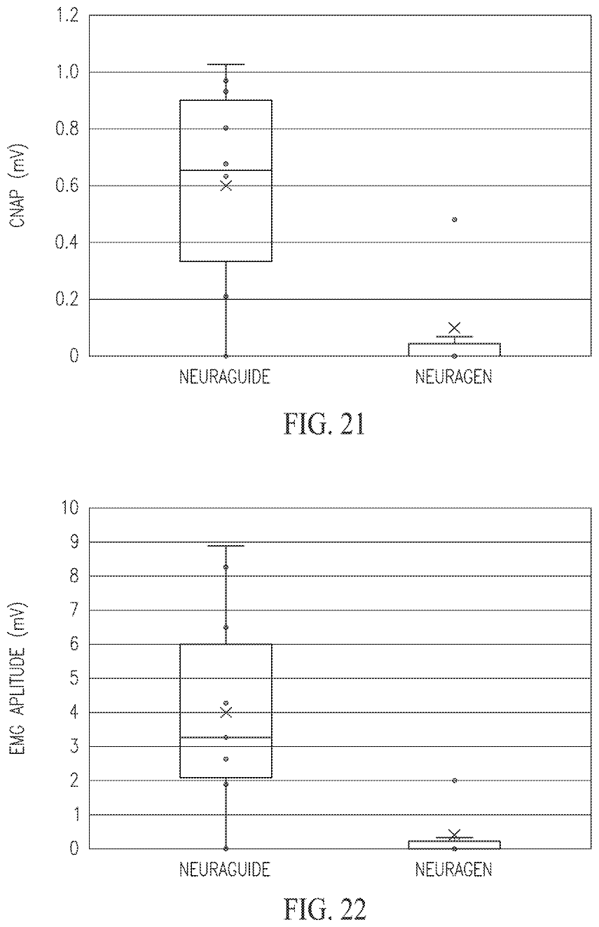

Electrophysiology: The compound nerve action potential (CNAP) for the NeuraGuide™ treatment group is compared to the NeuraGen® control group. As shown in FIG. 21, a measurable CNAP across the gap was present for 80% of the NeuraGuide™ treatment group, while only 33% of the control group showed a measurable CNAP. In addition, the CNAP magnitude which is indicative of the degree of functional recovery was six times higher than the control group (0.59 mV versus 0.09 mV). The difference between the two groups was statistically significant, p value=0.004. These results indicate that the NeuraGuide™ device with electrical stimulation enabled superior nerve growth across the gap as compared to the control group after 12 weeks. Similar conclusions can be made for the electromyography measurements between the two groups (FIG. 22).

6-week and 12-Week Observation: At 6 and 12 weeks post-surgery, the sciatic nerve transection bridged by the NeuraGuide™ device was observed. No sign of post-surgery inflammation was observed and the tissue surrounding the implanted scaffold appeared normal.

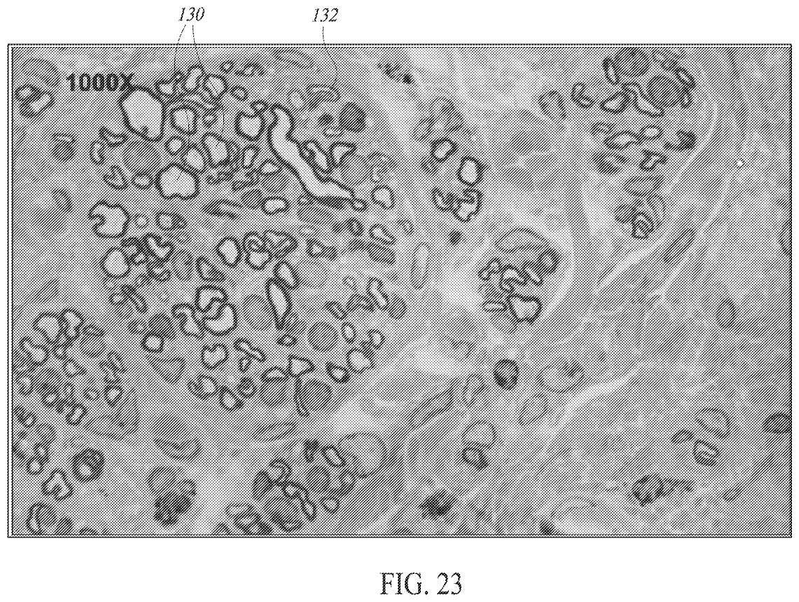

Histology: As shown in FIG. 23, a histological section taken near the distal end from an explanted NeuraGuide™ device shows numerous myelinated axons that have bridged the nerve gap. In this image, groups of axons (130) appear gathered in larger structures or fascicles (132), consistent with the rat sciatic nerve anatomy. There was no evidence of foreign body reaction, inflammation, or other features that could indicate issues with biocompatibility.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Patent Application No. 63/130,570, filed Dec. 24, 2020, which application is hereby incorporated by reference in its entirety.

1. An electrically conductive membrane comprising a biocompatible polymer layer and a conductive mesh of nanostructures, wherein the conductive mesh has a surface loading of the nanostructures in the range of 0.05 μg/cm² to 100 μg/cm², wherein the biocompatible polymer layer is 1 micron to 100 microns thick, and wherein the nanostructures are conductive or semiconductive nanostructures, or a combination thereof.

2. The electrically conductive membrane according to claim 1 wherein the membrane has a planar shape or is fabricated into a tubular shape, a spherical shape or a shape obtainable by deforming the said membrane.

3. The electrically conductive membrane according to claim 1 wherein the conductive mesh has a thickness of 5 nm to 500 nm.

4. (canceled)

5. The electrically conductive membrane according to claim 1 wherein the conductive mesh being at least partially incorporated into the biocompatible polymer layer.

6. The electrically conductive membrane according to claim 1 wherein the biocompatible polymer include one or more natural polymers selected from the group consisting of self-assembled polypeptides forming liquid crystal material, fibrillar polypeptides, collagens, fibrin, fibrinogen, fibronectin, laminin, silk, poly-L-lactic acid, elastin-like polypeptides, chitin, gelatin, glycosaminoglycans (GAGs), chitosan, sodium alginate, alginic acid, their derivatives, or a combination thereof, or the biocompatible polymer include one or more synthetic polymers selected from the group consisting of polyethylene glycol (PEG), polycaprolactone (PCL), polyglycolic acid (PGA), and poly(lactide-co-glycolide) (PLGA), hydroxypropyl cellulose (HPC), hydroxypropyl methylcellulose (HPMC), or a combination thereof.

7. (canceled)

8. The electrically conductive membrane according to claim 1 wherein the conductive nanostructures are formed from silver nanowires.

9. The electrically conductive membrane according to claim 1 having a resistance in the range from 1 ohm/sq to 10,000 ohm/sq.

10. An electrically conductive scaffold comprises an electrically conductive membrane of claim 1.

11. The electrically conductive scaffold of claim 10, wherein the electrically conductive membrane is rolled up into a tube having a hollow interior.

12. The electrically conductive scaffold of claim 11, wherein the tube further comprises one or more biocompatible polymer threads in the hollow interior.

13. (canceled)

14. An electrically conductive scaffold comprising a scaffold substrate coated with a conductive mesh of nanostructures, wherein the conductive mesh has a surface loading of the nanostructures in the range of 0.05 μg/cm² to 100 μg/cm².

15. An electrically conductive scaffold according to claim 14 wherein the conductive mesh has a thickness of 5 nm to 500 nm.

16. (canceled)

17. The electrically conductive scaffold according to claim 14 wherein the scaffold substrate has a tubular structure having an exterior surface and a hollow space defined by an interior surface, wherein the conductive mesh is on the interior surface of the tube, or on the exterior surface of the tube.

18. (canceled)

19. (canceled)

20. The electrically conductive scaffold according to claim 14 wherein the scaffold substrate is a thread or suture.

21. The electrically conductive scaffold according to claim 14, wherein the scaffold substrate is made of a biocompatible polymer layer.

22. The electrically conductive scaffold according to claim 21, wherein the biocompatible polymer include one or more natural polymers selected from the group consisting of self-assembled polypeptides forming liquid crystal material, fibrillar polypeptides, collagens, fibrin, fibrinogen, fibronectin, laminin, silk, poly-L-lactic acid, elastin-like polypeptides, chitin, gelatin, glycosaminoglycans (GAGs), chitosan, sodium alginate, alginic acid, their derivatives, or a combination thereof, or one or more synthetic polymers selected from the group consisting of polyethylene glycol (PEG), polycaprolactone (PCL), polyglycolic acid (PGA), and poly(lactide-co-glycolide) (PLGA), hydroxypropyl cellulose (HPC), hydroxypropyl methylcellulose (HPMC), or a combination thereof.

23. (canceled)

24. The electrically conductive scaffold according to claim 14, having a resistance in the range from 1 ohm/cm to 10,000 ohm/cm.

25. A method for promoting nerve repair or regrowth, the method comprising implanting the electrically conductive scaffold of claim 10 at a site of nerve damage.

26. The method of claim 25, wherein the electrically conductive scaffold provides electrical stimulation.

27. The method of claim 25, wherein the electrically conductive scaffold provides Joule heating.

28. (canceled)

29. (canceled)
